(19) United States
(12) Patent Application Publication
(10) Pub. No.: US 2026/0355250 A1
(43) Pub. Date: Nov. 12, 2026

(54) THIRD LINE TREATMENT FOR GVHD

(71) Applicant: MESOBLAST INTERNATIONAL SARL, Meyrin (CH)

(72) Inventors: Silviu ITESCU, Melbourne (AU); Jack HAYES, New York, NY (US)

(21) Appl. No.: 19/515,084

(22) PCT Filed: Aug. 28, 2024
(86) PCT No.: PCT/IB2024/058375

§ 371 Date: Feb. 27, 2026

Related U.S. Application Data
(60) Provisional application No. 63/579,213, filed on Aug. 28, 2023.

(51) Int. Cl. A61K 35/28; A61K 9/08; A61K 47/20; A61K 47/42; A61P 37/06

(52) U.S. Cl. CPC A61K 35/28; A61K 9/08; A61K 47/20; A61K 47/42; A61P 37/06

(57) ABSTRACT

The present disclosure relates to mesenchymal lineage precursor or stem cell mediated methods for treating severe Graft versus Host Disease (GvHD) in subjects that are refractory to at least two prior lines of therapy.

### TECHNICAL FIELD

The present disclosure relates to mesenchymal lineage precursor or stem cell mediated methods for treating severe Graft versus Host Disease (GvHD) in subjects that are refractory to at least two prior lines of therapy.

### BACKGROUND

Acute and chronic Graft versus Host Disease (GvHD) are immunological disorders that are the major cause of non-relapse mortality (NRM) after allogeneic stem cell transplantation. Acute GvHD affects 40% to 60% of patients and targets the skin, liver, and gastrointestinal tract.

GvHD remains a significant unmet clinical need, in particular in patients that have become refractory to currently approved therapies. Indeed, there are high levels of mortality in these patients. Clearly, there is an unmet therapeutic need in the art for treating GvHD, in particular in subjects at high risk of poor outcomes.

### SUMMARY

The present inventors have surprisingly identified that mesenchymal lineage precursor or stem cells are particularly effective at treating GvHD in subjects that have failed two prior lines of therapy. Indeed, marked improvements in survival have been observed in GvHD patients administered MSCs as a third line of therapy.

Accordingly, in a first example, the present disclosure relates to a method of treating Graft versus Host Disease (GvHD) in a human subject in need thereof, the method comprising administering to the subject a composition comprising mesenchymal lineage precursor or stem cells (MLPSCs), wherein the MLPSCs are administered to the subject as a third line of therapy.

In another example, the present disclosure relates to a method for treating Graft versus Host Disease (GvHD) in a human subject, the method comprising:
i) selecting a subject with GvHD that has failed two lines of therapy;
ii) administering to the subject a composition comprising mesenchymal lineage precursor or stem cells (MLPSCs), wherein the MLPSCs are administered to the subject as a third line of therapy.

In an example, subjects treated according to the methods disclosed herein are refractory to steroids and a second line of therapy. In an example, the second line of therapy is ruxolitinib. In an example, the second line of therapy is a biologic. Exemplary biologics include alemtuzumab, basiliximab or tocilizumab.

In an example, the MLPSCs in the composition inhibit IL-2Rα expression by ≥60%, wherein inhibition of IL-2Rα expression is determined by: obtaining a population of cells comprising culture expanded, cryopreserved and thawed MLPSCs; co-culturing the MLPSCs in a culture medium with a population of cells comprising T cells; and determining the level of inhibition of IL-2Rα expression.

In an example, treatment reduces the risk of mortality in the subject. For example, mortality may be 6-month non-relapse mortality (NRM).

In an example, the subject is a paediatric subject. In an example, the subject is ≥12 years of age.

In another example, the subject has acute Graft versus Host Disease (aGvHD). In another example, the subject is classified as one or more of the following:
Grade B, C, or D according to the IBMTR severity scale;
Grade II GvHD according to the Glucksberg severity scale;
Grade III/IV GvHD according to the Glucksberg severity scale;
Minnesota high risk GvHD.

In an example, the subject has chronic GvHD.

In an example, the subject has multi-organ involvement.

In an example, the subject has inflammatory bowel disease (IBD). In an example the IBD is Crohn's disease or ulcerative colitis. In an example, the IBD is Crohn's disease. In an example, the Crohn's disease presents in the rectum and/or colon of the subject. In an example, the subjects IBD is refractory to one or more of adalimumab, certolizumab pegol, vedolizumab or Ustekinumab.

In an example, treatment increases the probability of the subject surviving for at least 28 days after initiation of treatment. In an example, treatment increases the probability of the subject surviving for at least 100 days after initiation of treatment, preferably at least 180 days. In an example, treatment increases the probability of the subject surviving for at least 100 days after initiation of treatment. In an example, the subject's probability of survival is increased relative to a subject who does not receive MLPSCs. In an example, the probability of the subject surviving is greater than 40%, greater than 50%, or, preferably, greater than 60% after initiation of treatment. In an example, the probability of the subject surviving is greater than 40% after initiation of treatment. In an example, the probability of the subject surviving is greater than 50%, after initiation of treatment. In an example, the probability of the subject surviving is greater than 60% after initiation of treatment. In an example, the probability of the subject surviving is between about 30% and about 80% after initiation of treatment. In an example, the probability of the subject surviving is between about 40% and about 70% after initiation of treatment. In an example, the probability of the subject surviving is between about 50% and about 65% after initiation of treatment. In an example, the probability of the subject surviving is between about 60% and about 70% after initiation of treatment.

In an example, the MLPSCs are culture expanded from a population of MLPSCs that are STRO-1+. For example, the population of MLPSCs that are STRO-1+ may comprise about 0.1% to 75% STRO-1+ cells.

In an example, the MLPSCs are mesenchymal stem cells (MSCs).

In an example, the composition further comprises Plasma-Lyte A, dimethyl sulfoxide (DMSO), human serum albumin (HSA). In an example, the composition comprises greater than 6.68×10⁶ viable cells/mL. In an example, the composition is administered intravenously.

In an example, the subject receives at least two doses. In an example, the treatment comprises administering two doses a week for four weeks. In another example, treatment comprises administering two doses a week for eight weeks. In an example, the doses are administered at least three days apart.

### BRIEF DESCRIPTION OF DRAWINGS

FIG. 1: Kaplan-Meier survival estimates through day 100 for 71 patients with acute GVHD treated with MSCs as third-line after failure to corticosteroids and second-line agents, including ruxolitinib.

FIG. 2: Kaplan-Meier survival estimates through day 100 for 71 patients with acute GVHD treated with MSCs as third line and stratified on the basis of Day 28 response or non-response.

FIG. 3: Kaplan-Meier survival estimates through day 100 for MSCs provided as second-line (n=54) or third-line (n=35) in children under age of 18 with steroid-refractory acute GvHD.

### DETAILED DESCRIPTION

### General Techniques and Definitions

Unless specifically defined otherwise, all technical and scientific terms used herein shall be taken to have the same meaning as commonly understood by one of ordinary skill in the art (e.g., in cell culture, molecular biology, stem cell culture, immunology, clinical studies, general medicine, and biochemistry).

Unless otherwise indicated, cell culture techniques and assays utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

As used herein, the term “about”, unless stated to the contrary, refers to +/−10%, more preferably +/−5%, of the designated value.

The terms “level” and “amount” are used to define the amount of a particular substance in a cell preparation. For example, a particular concentration, weight, percentage (e.g. v/v %) or ratio can be used to define the level of a particular substance. In an example, the level is expressed in terms of how much of a particular marker is expressed by cells disclosed herein under culture conditions. In an example, expression represents cell surface expression. In another example, the level is expressed in terms of how much of a particular marker is released from cells described herein into their culture medium under culture conditions.

In an example, the level of a particular marker is determined under culture conditions. The term “culture conditions” is used to refer to cells growing in culture. In an example, culture conditions refers to an actively dividing population of cells. Such cells may, in an example, be in exponential growth phase. In an example, the cells may be in a stationary phase.

In an example, culture conditions encompass co-culture of an MLPSC population disclosed herein and a second cell population such as a population which comprises peripheral blood mononuclear cells (PBMC). In an example, co-culture comprises culturing an MLPSC population disclosed herein and a population of activated PBMC. For example, PBMC can be activated using anti-CD3 and anti-CD28 antibodies before co-culture with an MLSPC population disclosed herein.

In an example, “culture conditions” comprises co-culturing MLPSCs and T cells at a ratio of about 1 MLPSC: 2 T cells, or less. For example, 1:3, 1:4, 1:5, 1:10, 1:20, 1:30, 1:40, 1:50, 1:60, 1:70 1:80, 1:90, or 1 MLPSC: 100 T cells, or less. In this example, the level of IL2-RA inhibition is determined after about 30 to 84 hours of cell culture under culture conditions.

In an example, the level of a particular marker can be determined by taking a sample of cell culture media and measuring the level of marker in the sample. In another example, the level of a particular marker can be determined by taking a sample of cells and measuring the level of the marker in the cell lysate. Those of skill in the art will appreciate that secreted markers can be measured by sampling the culture media while markers expressed on the surface of the cell may be measured by assessing a sample of cell lysate. In an example, the sample is taken when the cells are in exponential growth phase. In an example, the sample is taken after at least two or three days in culture. In another example, the sample is taken after about 30 to 84 hours of co-culture. In an example, the sample is taken from a co-culture of MLPSCs and activated PBMCs. In this example, the cell sample can be lysed and the level of a marker can be determined. For example, the level of IL2-RA may be determined using various methods such as an enzyme-linked immunosorbent assay (ELISA) based method. In an example, the ELISA comprises:
(i) adding sample diluent to each well of a microplate precoated with a monoclonal antibody specific for IL2-RA;
(ii) adding a co-cultured sample to a well of a microplate precoated with a monoclonal antibody specific for IL2-RA;
(iii) incubating the microplate for sufficient time to allow for the monoclonal antibody specific for IL2-RA to specifically bind to any IL2-RA in the sample;
(iv) washing the microplate;
(v) adding IL2-RA conjugate to the well;
(vi) incubating the microplate for sufficient time to allow the conjugate to specifically bind to any captured IL2-RA;
(vii) washing the microplate;
(viii) adding a substrate solution to the well;
(ix) incubating the microplate for sufficient time for colour development;
(x) adding a stop solution to the well;
(xi) reading optical density on a microplate reader set to 450 nm with wavelength correction at 570 nm;
(xii) determining the concentration of IL2-RA.

In another example, the level of IL2-RA is determined using fluorescence-activated cell sorting (FACS) using appropriate antibodies such as anti-CD25. Further antibodies may also be employed if required to distinguish CD25+ cell types.

In an example, the level of IL2-RA in co-culture is compared with the level of IL2-RA in cultured population of activated PBMC. Levels of IL2-RA are subsequently compared to provide a level of IL2-RA inhibition in co-culture. For example, the level of IL2-RA may be inhibited at least 60% in co-culture (i.e. MLPSC: activated PBMC) relative single culture (activated PBMC alone). Those of skill in the art will appreciate other suitable methods of determining level of IL2-RA under culture conditions.

Culture expanding cells from a cryopreserved intermediate means thawing cells subject to cryogenic freezing and in vitro culturing under conditions suitable for growth of the cells.

In an example, the “level” or “amount” of a particular marker such as IL2-RA or TNF-R1 is determined before cells have been cryopreserved. For example, the level may be determined after the first 2 to 5 passages of the cells. In another example, the level or amount of a particular marker is determined after a first cryopreservation of cells. In another example, the level is determined after a second cryopreservation of cells. For example, cells may be culture expanded to provide an intermediate, cryopreserved, defrosted before being re-seeded in culture so that the level of a particular marker can be determined under culture conditions and, cryopreserved a second time.

By “isolated” or “purified” it is meant a cell which has been separated from at least some components of its natural environment. This term includes gross physical separation of the cells from its natural environment (e.g. removal from a donor). The term “isolated” includes alteration of the cell's relationship with the neighboring cells with which it is in direct by, for example, dissociation. The term “isolated” does not refer to a cell which is in a tissue section. When used to refer to the population of cells, the term “isolated” includes populations of cells which result from proliferation of the isolated cells of the disclosure.

The terms “passage”, “passaging” or “sub-culture” are used in the context of the present disclosure to refer to known cell culture techniques that are used to keep cells alive and growing under cultured conditions for extended periods of time so that cell numbers can continually increase. The degree of sub-culturing a cell line has undergone is often expressed as “passage number,” which is generally used to refer to the number of times cells have been sub-cultured. In an example, one passage comprises removing non-adherent cells and leaving adherent mesenchymal lineage precursor or stem cells. Such mesenchymal lineage precursor or stem cells can then be dissociated from the substrate or flask (e.g., by using a protease such as trypsin or collagenase), media can be added, optional washing (e.g., by centrifugation) may be performed, and then the mesenchymal lineage precursor or stem cells can be re-plated or reseeded to one or more culture vessels containing a greater surface area in total. The mesenchymal lineage precursor or stem cells can then continue to expand in culture. In another example, methods of removing non-adherent cells include steps of non-enzymatic treatment (e.g., with EDTA). In an example, mesenchymal lineage precursor or stem cells are passaged at or near confluence (e.g., about 75% to about 95% confluence). In an example, the mesenchymal lineage precursor or stem cells are seeded at a concentration of about 10%, about 15%, or about 20% cells/ml of culture medium.

The term “medium” or “media” as used in the context of the present disclosure, includes the components of the environment surrounding cells in culture. It is envisaged that the media contributes to and/or provides the conditions suitable to allow cells to grow. Media may be solid, liquid, gaseous or a mixture of phases and materials. Media can include liquid growth media as well as liquid media that do not sustain cell growth. Exemplary gaseous media include the gaseous phase that cells growing on a petri dish or other solid or semisolid support are exposed to.

In an example, the present disclosure encompasses selecting certain subjects with GvHD for treatment with a MLPSC composition disclosed herein. In an example, subjects with GvHD that have failed two lines of therapy are selected for treatment. For example, GvHD subjects that have are refractory to steroids and a second line of therapy are selected for treatment. In an example, GvHD subjects that are refractory to steroids and ruxolitinib are selected for treatment.

In an example, the subject has a reduced risk of mortality after treatment with MLPSCs as third line therapy. In one example, the reduced risk may be relative to risk of mortality in a subject that has not been administered MLPSCs. In another example, the reduced risk is relative to the risk of mortality in the same subject before being administered MLPSCs.

In an example, mortality is “non-relapse mortality”. The term “non-relapse mortality” refers to death without recurrent or progressive disease. In an example, the subject has a reduced risk of 6 month non-relapse mortality. In another example, the subject has a reduced risk of 1 year non-relapse mortality.

In an example, treatment increases subject survival. In an example, treatment increases the probability of a subject surviving for at least 100 days after initiation of treatment. In another example, treatment increases the probability of a subject surviving for at least 180 days after initiation of treatment. In an example, the increased probability is determined relative to a subject that is not treated with MLPSCs.

The term “subject” as used herein refers to a human subject. For example, the subject can be an adult. In another example, the subject can be a child. In another example, the subject can be an adolescent. In an example, the subject can be a paediatric subject. Paediatric is the branch of medicine that involves the medical care of infants, children, and adolescents. In one example, the subject is less than 25 years of age. In another example, the subject is less than 21 years of age. In another example, the subject is less than 18 years of age. In an example, the paediatric subject can range in age from birth to 17 years old.

Terms such as “subject”, “patient” or “individual” are terms that can, in context, be used interchangeably in the present disclosure.

Subjects treated according to the methods disclosed herein are refractory to certain therapies. The term “refractory” is used in the context of the present disclosure to refer GvHD that has stopped responding to a particular treatment. For example, subjects with steroid refractory GvHD have GvHD that has stopped responding to treatment with the steroid. In an example, a subject is considered refractory to a line of therapy when their GvHD has worsened within 3 days of therapy. In another example, a subject is considered refractory to a line of therapy when their GvHD has worsened within 7 days of therapy.

Subjects may be refractory to multiple lines of therapy. For example, GvHD subjects may be refractory to steroid therapy and a second line of therapy. “Lines of therapy” is used in the context of the present disclosure (and clinically) to describe the order in which different therapies are given to patients with GvHD as their disease progresses. For example, subjects treated herein may be refractory to a first line of steroid therapy and a second line of therapy. In an example, the subject's GvHD may become refractory to the steroid before the second line of therapy is administered. In another example, the subject may be treated with a steroid in combination with a second line of therapy, becoming refractory to both lines of therapy before being treated with MLPSCs disclosed herein.

“Therapeutic efficacy” is used in the context of the present disclosure to refer to MLPSCs and compositions disclosed herein that can treat, inhibit and/or prevent disease. For example, therapeutically effective MLPSCs and compositions disclosed herein can treat, inhibit and/or prevent GvHD in patients that have failed two prior lines of therapy. In an example, therapeutically effective MLPSCs and compositions disclosed herein increase 100 day survival in subjects with GvHD that have failed two prior lines of therapy.

The term “clinically proven” (used independently or to modify the term “effective”) shall mean that efficacy has been proven by a clinical trial wherein the clinical trial has met the approval standards of U.S. Food and Drug Administration, EMEA or a corresponding national regulatory agency. For example, the clinical study may be an adequately sized, randomized, double-blinded study used to clinically prove the effects of the composition. In an example, a clinically proven effective amount is an amount shown by a clinical trial to meet a specified endpoint. In an example, the end point is protection against death. Put another way, the end point increases survival. For example, 100 day survival may be increased when administering treatment according to the present disclosure.

Accordingly, the terms “clinically proven efficacy” and “clinically proven effective” can be used in the context of the present disclosure to refer to a dose, dosage regimen, treatment or method disclosed herein. Efficacy can be measured based on change in the course of the disease in response to administering a composition disclosed herein. For example, a composition of the disclosure is administered to a subject in an amount and for a time sufficient to induce an improvement, preferably a sustained improvement, in at least one indicator that reflects the severity of GvHD. Various indicators that reflect the severity of the disease can be assessed for determining whether the amount and time of the treatment is sufficient. Such indicators include, for example, clinically recognized indicators of disease severity or symptoms. In an example, the degree of improvement is determined by a physician, who can make this determination based on signs, symptoms, or other test results (e.g. MAP; Reg3α and ST2 levels; skin % BSA; mouth score; eye score of at least one point; skin features score; gastrointestinal tract score; liver score; lung symptom score; lung FEV1 score; joints and fascia score; and/or genital tract score).

In an example, a clinically proven effective amount improves patient survival. In another example, a clinically proven effective amount reduces a subjects risk of mortality, for example non-relapse mortality. In another example, a clinically proven effective amount increases 100 day survival. In an example, methods of the disclosure administer a clinically proven effective amount of a composition disclosed herein. In an example, a clinically proven effective amount is characterised by overall survival>40% 100 days after treatment. In an example, a clinically proven effective amount is characterised by overall survival>50% 100 days after treatment. In an example, a clinically proven effective amount is characterised by overall survival>55% 100 days after treatment. In an example, a clinically proven effective amount is characterised by overall survival between 40% and 80% 100 days after treatment. In an example, a clinically proven effective amount is characterised by an overall response rate>40%. In an example, a clinically proven effective amount is characterised by an overall response rate>50%.

In an example, compositions of the disclosure comprise genetically unmodified mesenchymal precursor lineage or stem cells. As used herein, the term “genetically unmodified” refers to cells that have not been modified by transfection with a nucleic acid. For the avoidance of doubt, in the context of the present disclosure a mesenchymal lineage precursor or stem cell transfected with a nucleic acid encoding a protein would be considered genetically modified.

The term “total dose” is used in the context of the present disclosure to refer to the total number of cells received by the subject treated according to the present disclosure. In an example, the total dose consists of one administration of cells. In another example, the total dose consists of two administrations of cells. In another example, the total dose consists of three administrations of cells. In another example, the total dose consists of four or more administrations of cells. For example, the total dose can consist of two to four administrations of cells.

The term “conditioned media” is used in the context of the present disclosure to refer to media obtained from MLPSCs under culture conditions. Such media contains the MLPSC secretome, proteins shed from the surface of MLPSCs and, other particles such as extracellular vesicles. Conditioned media of the disclosure contains extracellular vesicles and/or secreted metabolites such as prostaglandin E2. In certain examples, the present disclosure relates to administration of extracellular vesicles such as exosomes that have been obtained from conditioned media obtained from MLPSCs under culture conditions. In an example, the conditioned media is obtained when the MLPSCs are in exponential growth phase. In an example, the conditioned media is obtained after at least two or three days in culture. In another example, the conditioned media is obtained after about 30 to 84 hours of culture.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Those skilled in the art will appreciate that the disclosure described herein is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific embodiments described herein, which are intended for the purpose of exemplification only. Functionally-equivalent products, compositions and methods are clearly within the scope of the disclosure, as described herein.

Any example disclosed herein shall be taken to apply mutatis mutandis to any other example unless specifically stated otherwise.

### Graft Versus Host Disease

Methods of the present disclosure encompass treatment of Graft versus Host Disease (GvHD). GvHD is an immunological disorder that is the major factor that limits the success and availability of allogeneic bone marrow or stem cell transplantation. GvHD occurs in acute (aGvHD) or chronic (cGvHD) forms. Acute GvHD usually manifests within 100 days following bone marrow or stem cell transplantation. Chronic GvHD generally manifests later than aGvHD (>100 days post transplantation) and has some features of autoimmune diseases. It may develop either de novo, following resolution of aGvHD or as an extension of aGvHD. Chronic GvHD can cause multiple, often debilitating symptoms, including widespread skin rashes, painful mouth ulcers, shortness of breath, and limb and joint pain.

The present inventors have identified that MLPSC compositions of the disclosure are surprisingly effective as a third line therapy for GvHD. The surprising nature of the present inventors findings stems, at least in part, from the exceptionally poor prognosis in patients with GvHD that is refractory to two prior lines of therapy. Accordingly, methods of the present disclosure are effective in a subset of patients with GvHD. These subjects are refractory to two prior lines of therapy. In an example, these subjects have steroid refractory GvHD. For example, GvHD subjects can be refractory to a steroid and a second line of therapy. In an example, the subject is refractory to a steroid and ruxolitinib. In another example, the subject is refractory to a steroid and a biologic. In an example, the biologic is alemtuzumab, basiliximab or tocilizumab. In an example, the steroid is a corticosteroid. In another example, the steroid is a glucocorticoid. In another example, the steroid is prednisone. In an example, GvHD subjects are refractory to a corticosteroid and a second line of therapy such as ruxolitinib. In an example, GvHD subjects are refractory to prednisone and a second line of therapy such as ruxolitinib. In an example, the subject has GvHD that is refractory to steroid therapy and the subject has ruxolitinib intolerance.

In an example, the present disclosure encompasses treatment of patients with acute GvHD. In another example, the present disclosure encompasses treatment of patients with chronic GvHD. In an example, the GvHD presents in the stomach and/or gut of the subject as damage to these organs. In this example, various sites of inflammation can occur along the stomach and/or gut and, in certain examples, it may be preferable to administer treatment directly to one or more of these sites of inflammation.

In an example, the present disclosure encompasses use of MLPSCs as a third line of therapy for treating subjects that have severe GvHD. In an example, the severe GvHD is determined based on the subject's MAGIC Algorithm Probability (MAP). MAP is a validated analysis that combines the serum concentrations of two biomarkers, Regenerating islet-derived protein 3 alpha (Reg3α) and soluble interleukin 1 receptor-like 1 (ST2), into a single value that predicts long-term outcomes such as response to therapy after 28 days and 6-month non-relapse mortality (NRM). MAP is calculated by:

log 10[−log 10 (1−\hat{p})]=−11.263+1.844 (log 10ST2)+0.577 (log 10REG3α),

where \hat{p} is the predicted probability of 6-month NRM (Hartwell et al.; Major-Monfried et al.). More severe GvHD is associated with higher MAP scores. Accordingly, in an example, “severe GvHD” according to the present disclosure is defined based on MAP. In an example, the subject has a MAP greater than 0.16, preferably greater than 0.2, more preferably greater than 0.25. A MAP score of ≥0.291 is a validated threshold that identifies GvHD patients with the most severe disease (Hartwell et al.; Major-Monfried et al.) For example, patients with a MAP score of ≥0.291 are at high risk of non-response to treatment and death. Accordingly, in an example, the subject has a MAP≥0.29. In an example, the subject has a MAP≥0.291. In another example, subject has a MAP between 0.15 and 0.8. In another example, the subject has a MAP between 0.16 and 0.7. In another example, subject has a MAP between 0.2 and 0.7. In another example, subject has a MAP between 0.29 and 0.6.

In one example, subjects with severe GvHD as determined by high MAP have a low probability of achieving a clinical response to primary therapy within 28 days and/or a high risk of 6 month non-relapse mortality. Accordingly, in one example, the subject has a 6 month non-relapse mortality risk of ≥70%. In another example, the subject has a 6 month non-relapse mortality risk of ≥80%. In another example, the subject has a 6 month non-relapse mortality risk of ≥90%. In these examples, the subject can have a MAP≥0.29.

High MAP is indicative of severe gastrointestinal (GI) crypt damage. GI crypts are glands found in between villi in the intestinal epithelium lining of the small intestine and large intestine. GI crypt cells provide stem cells for renewal of the intestinal epithelium. For example, subjects with a MAP of ≥0.29 generally have severe GI crypt damage. In an example, crypt damage is determined by histological assessment. For example, a biopsy of gastrointestinal tissue is taken during an endoscopy or colonoscopy which is then histologically assessed for cellular damage and inflammation. Accordingly, in an example, subjects treated according to the present disclosure can have severe GI crypt damage.

As outlined above, MAP is determined based on Reg3α and ST2 levels. Accordingly, in an example, serum concentrations of Reg3α and ST2 are determined in a subject. In an example, the level of ST2 and Reg3α are quantified in the same sample. In another example, the level of ST2 and Reg3α are quantified in separate samples. In an example, multiple samples are obtained periodically, wherein ST2 and Reg3α are quantified in each sample so that a subject's MAP can be monitored over time.

GvHD severity can also be graded by patterns of organ involvement and clinical performance status. Multi-organ involvement includes skin rash, liver involvement, and/or gastrointestinal (GI) involvement. Examples of skin rash, liver involvement, and GI involvement are provided in Table 1 and Table 2. In an example, the subject has GvHD with multi-organ involvement. In an example, severe GvHD is graded according to the Glucksberg scale (Glucksberg et al, 1974; Thomas et al, 1975) (Table 1). For example, the subject can have Grade II GvHD or Grade III/IV GvHD according to the Glucksberg scale. In one example, the subject has Grade II GvHD. In another example, the subject has Grade III/IV GvHD.

In another example, severe GvHD is graded according to IBMTR Severity Index (Table 2) (Rowlings et al., 1997). In one example, the subject has Grade B, Grade C, or Grade D GvHD according to the IBMTR severity scale.

In another example, the subject has Minnesota high risk GvHD. Minnesota high risk acute GvHD is defined as either skin stage 4; lower GI stage 3-4 or liver stage 3-4; or skin stage 3+ and either lower GI 2-4 or liver stage 2-4 GvHD (MacMillan et al., 2015). In each of these examples, the subject can also have a high MAP score. For example, the subject can have a MAP score≥0.29.

TABLE 1
Glucksberg clinical stage and grade of
acute GvHD (Rowlings et al., 1997).

| Stage | Skin | Liver | Intestinal tract |
|---|---|---|---|
| 1 | Maculopapular | Bilirubin 34-50 | >500 ml |
| | rash <25% of body | μmol/l | diarrhoea/d |
| | surface | | |
| 2 | Maculopapular | Bilirubin 51-102 | >1000 ml |
| | rash 25-50% of body | μmol/l | diarrhoea/d |
| | surface | | |
| 3 | Generalised | Bilirubin 103-225 | >1500 ml |
| | erythroderma | μmol/l | diarrhoea/d |
| 4 | Generalised | Bilirubin >225 | Severe abdominal |
| | erythroderma with | μmol/l | pain, with or |
| | bullous formation | | without ileus |
| | and desquamation | | |

| Grade | Degree of organ involvement |
|---|---|
| I | Stage 1-2 skin rash: no gut involvement: no liver involvement: |
| | no decrease in clinical performance |
| II | Stage 1-3 skin rash: stage 1 gut involvement or stage 1 liver |
| | involvement (or both): mild decrease in clinical performance |
| III | Stage 2-3 skin rash: stage 2-3 gut involvement or stage 2-4 liver |
| | involvement (or both): marked decrease in clinical performance |
| IV | Similar to Grade III with stage-4 organ involvement and extreme |
| | decrease in clinical performance |

TABLE 2
Criteria for IBMTR Severity Index for
acute GvHD (Rowlings et al., 1997).

| | | | | Liver | | | Gastrointestinal | |
|---|---|---|---|---|---|---|---|---|
| | Skin | | | involvement | | | invovlement | |
| | involvement | | | | Total | | | Volume of |
| In- | Stage | Extent | | Stage | bilirubin | | Stage | diarrhoea |
| dex* | (max) | of rash | | (max) | (μmol/l) | | (max) | (ml/d) |
| A | 1 | <25% | | 0 | <34 | | 0 | <500 |
| B | 2 | 25-50% | or | 1-2 | 34-102 | or | 1-2 | 550-1500 |
| C | 3 | >50% | or | 3 | 103-255 | or | 3 | >1500 |
| D | 4 | Bullae | or | 4 | >255 | or | 4 | Severe |
| | | | | | | | | pain and |
| | | | | | | | | ileus |

*Assign Index based on maximum in an individual organ system

In an example, patients with GvHD that are treated according to the disclosure have inflammatory bowel disease (IBD). For example, the GvHD may be accompanied by Crohn's disease or ulcerative colitis. In an example, the GvHD is accompanied by Crohn's disease. In an example, the Crohn's disease presents in the rectum and/or colon of the subject. In an example, the subjects IBD is refractory to one or more of lines of therapy. In an example, the subjects IBD is refractory to treatment with a biologic. In an example, the subjects IBD is refractory to treatment with adalimumab, certolizumab pegol, vedolizumab or Ustekinumab.

### Selecting a Patient

In an example, the present disclosure encompasses selecting certain subjects with GvHD for treatment with MLPSCs. In an example, subjects with GvHD that have failed two lines of therapy are selected for treatment. For example, methods of selecting a subject for treatment comprises the steps of i) selecting a subject with GvHD that has failed two lines of therapy for treatment. In an example, the selection method comprises 1) selecting a subject with GvHD that is refractory to steroids and a second line of therapy. In an example, the selection method comprises 1) selecting a subject with GvHD that is refractory to a steroid and ruxolitinib. In an example, the selection method comprises 1) selecting a subject with GvHD that is refractory to prednisone and ruxolitinib.

### Treatment Response

Methods of the present disclosure relate to a third line treatment of GvHD. As used herein, the terms “treating”, “treat”, “treatment”, “reducing progression” include administering a population of MLPSCs and/or progeny thereof and/or soluble factors derived therefrom and/or extracellular vesicles derived therefrom to thereby reduce or eliminate at least one symptom of GvHD.

In an example, treatment reduces a subject's ST2 and/or Reg3α level. For example, a subject's ST2 and/or Reg3α level is reduced relative to the subject's baseline level. In an example, treatment reduces a subject's ST2 level. For example, the subject's ST2 level is reduced relative to the subject's baseline ST2 level. In another example, treatment reduces a subject's Reg3α level. For example, the subject's Reg3α level is reduced relative to the subject's baseline Reg3α level. In another example, treatment reduces a subject's MAP. For example, the subject's MAP is reduced relative to the subject's baseline level. In an example, the subject's MAP is reduced to <0.29. In another example, the subject's MAP is reduced to <0.291. In these examples, treatment decreases the subject's MAP by day 28. In another example, treatment decreases the subject's MAP by day 100. In another example, treatment decreases the subject's MAP by day 160. In another example, treatment decreases the subject's MAP by day 180. In an example, the decrease in MAP is sustained for at least 1 month. In another example, the decrease in MAP is sustained for at least 3 months.

In an example, treatment decreases the level of one or more inflammatory biomarkers such as ELAFIN, sIL2-ra, TNFR1, IL-8 and HGF. For example, treatment can reduce the level of ELAFIN. In another example, treatment can reduce the level of sIL2-ra. In another example, treatment can reduce the level of TNFR1. In another example, treatment can reduce the level of IL-8. In example, the level of inflammatory biomarker is reduced between day 50 and 200 after treatment. In an example, the level of inflammatory biomarker is reduced between day 100 and 200 after treatment. In another example, the level of inflammatory biomarker is reduced between by day 100 after treatment. In another example, the level of inflammatory biomarker is reduced between by day 200 after treatment.

In another example, the reduction in the subject's inflammatory biomarker is sustained at day 100, at day 160, at day 180. For example, a reduction in one or more of ELAFIN, sIL2-ra, TNFR1, IL-8 and HGF can be observed at day 100 and sustained at day 180.

In an example, a decrease in the level of one or more of ELAFIN, sIL2-ra, TNFR1, IL-8 and HGF accompanies a decrease in the subjects MAP score.

In an example, treatment reduces GI crypt damage in a subject. For example, a subject's GI crypt damage is reduced relative to the subject's baseline level. In these examples, a reduced MAP is indicative of reduced crypt damage. In another example, reduced crypt damage is determined by histological assessment of a gastrointestinal tissue biopsy obtained by endoscopy or colonoscopy. For example, treatment reduces histological signs of cellular damage and inflammation in gastrointestinal crypts.

In an example, treatment is observed at day 28.

As used herein, the term “response” means response to therapy. In an example, a subject is considered to have had a response if they have an improvement in at least one organ without progression in any other organs and if additional therapy was not required. In another example, a subject is considered not to have had a response if they had stable or progressive GvHD or if the subsequent addition of a further line of therapy is required. In this example, a subject who does not have a response is a non-responder.

In an example, treatment induces a partial response. In an example, the partial response is induced at least 28 after treatment is initiated. In an example, the partial response is induced 28 days after treatment is initiated. In an example, the partial response is induced at least 30 days after treatment is initiated. In an example, the partial response is induced at least 2 months after treatment is initiated. In another example, the partial response is induced at least 3 months after treatment is initiated. In another example, the partial response is induced within 3 months. In another example, the partial response is induced 28 to 56 days after treatment is initiated. In another example, the partial response is induced 100 days after treatment is initiated. In another example, the partial response is induced 160 days after treatment is initiated. In another example, the partial response is induced 180 days after treatment is initiated.

In another example, the partial response is induced after eight doses. In another example, the partial response is induced after administering does twice weekly for four weeks. In another example, the partial response is induced after three doses or more. In an example, a partial response is characterized by one or more or all of:
Reduction in Skin % BSA score of at least one point;
Reduction in mouth score of at least one point;
Reduction in eye score of at least one point;
Reduction in skin features score of at least one point;
Reduction in gastrointestinal tract score of at least one point;
Reduction in liver score of at least one point;
Reduction in lung symptom score of at least one point;
Reduction in lung FEV1 score of at least one point;
Reduction in joints and fascia score of at least one point;
Reduction in genital tract score of at least one point.

In an example, a partial response is characterized by a reduction in Skin % BSA score of at least one point. In another example, a partial response is characterized by a reduction in mouth score of at least one point. In another example, a partial response is characterized by a reduction in eye score of at least one point. In these examples, scores can be obtained using the NIH Consensus Criteria 2014 for GvHD.

In another example, a partial response is characterized by one or more or all of:
Reduction in Skin % BSA score of at least one point;
Reduction in mouth score of at least one point;
Reduction in eye score of at least one point.

There are various classification systems for characterizing GvHD (Lee, S., (2017) Blood, 129(1): 30-37). In an example, the NIH Consensus Criteria 2014 can be used for scoring outcomes disclosed herein (Jagasia et al., (2015) Biol Blood Marrow Transplant, 21:389-401). The components of the NIH Consensus Criteria 2014 are shown in the following table:

TABLE 3
Organ Scoring of GvHD

| Organ | Score 0 | Score 1 | Score 2 | Score 3 |
|---|---|---|---|---|
| Skin % BSA¹ | No BSA | 1-18% BSA | 19-50% BSA | >50% BSA |
| | involved | | | |
| Skin Features | No sclerotic | N/A | Superficial | Deep sclerotic |
| | features | | sclerotic features, | features; |
| | | | but not | “hidebound”; |
| | | | “hidebound” | impaired mobility; |
| | | | | ulceration |
| Mouth | No symptoms | Mild | Moderate | Severe symptoms |
| | | symptoms | symptoms with | with disease signs |
| | | with disease | disease signs with | with major limitation |
| | | signs but not | partial limitation of | of oral intake |
| | | limiting oral | oral intake | |
| | | intake | | |
| | | significantly | | |
| Eyes | No symptoms | Mild dry eye | Moderate dry eye | Severe dry eye |
| | | symptoms not | symptoms partially | symptoms |
| | | affecting | affecting ADL | significantly |
| | | ADL | (requiring lubricant | affecting ADL |
| | | (requirement | drops >3×/day or | (special eyewear to |
| | | of lubricant | punctal plugs) | relieve pain) OR |
| | | drops ≤3×/day) | WITHOUT new | unable to work |
| | | | vision impairment | because of ocular |
| | | | due to | symptoms OR loss |
| | | | keratoconjunctivitis | of vision due to |
| | | | sicca (KCS) | keratoconjunctivitis |
| | | | | sicca (KCS) |
| GI Tract | No symptoms | Symptoms | Symptoms | Symptoms associated |
| | | without | associated with | with significant |
| | | significant | mild to moderate | weight loss (>15%) |
| | | weight loss | weight loss (5- | within 3 months, |
| | | (<5%) | 15%) within 3 | requires nutritional |
| | | | months OR | supplement for most |
| | | | moderate diarrhea | calorie needs OR |
| | | | without significant | esophageal dilation |
| | | | interference with | OR severe diarrhea |
| | | | daily living | with significant |
| | | | | interference with |
| | | | | daily living |
| Liver | Normal total | Normal total | Elevated total | Elevated total |
| | bilirubin and | bilirubin with | bilirubin but ≤3 | bilirubin but >3 |
| | ALT or | ALT ≥3 to | mg/dL or ALT >5 × | mg/dL |
| | AP <3 × ULN | 5 × ULN or | ULN | |
| | | AP ≥3 × ULN | | |
| Lungs | No symptoms | Mild | Moderate | Severe symptoms |
| Symptom | | symptoms | symptoms (SOB | (SOB at rests; |
| Score | | (SOB after | after walking on | requires O2) |
| | | climbing one | flat ground) | |
| | | flight of | | |
| | | steps) | | |
| Lungs Lung | FEV1 ≥80% | FEV1 60-79% | FEV1 40-59% | FEV1 ≤39% |
| Score | | | | |
| Joints and | No symptoms | Mild | Tightness of arms | Contractures WITH |
| Fascia | | tightness of | or legs OR joint | significant decrease |
| | | arms and | contractures, | of range of motion |
| | | legs, normal | erythema thought | AND significant |
| | | or mild | to be due to | limitation of ADL |
| | | decreased | fasciitis, moderate | (unable to tie shoes, |
| | | range of | decrease of range | button shirts, dress |
| | | motion AND | of motion AND | shelf, etc.) |
| | | not affecting | mild to moderate | |
| | | ADL | limitation of ADL | |
| Genital Tract² | No signs | Mild signs | Moderate signs and | Severe signs with or |
| | | and females | may have signs of | without symptoms |
| | | with or | discomfort on | |
| | | without | exam | |
| | | discomfort on | | |
| | | exam | | |
| Other | No GvHD | Mild | Moderate | Severe |
| features³ | | | | |

In an example, a partial response is a decrease of ≥1 point on the organ-specific NIH Consensus Criteria 2014 score from the Table above. Accordingly, in an example, treatment induces ≥1 point decrease in Skin % BSA score. In another example, treatment induces ≥1 point decrease in mouth score. In another example, treatment induces ≥1 point decrease in eye score. In another example, treatment induces ≥1 point decrease in skin features score. In another example, treatment induces ≥1 point decrease in gastrointestinal tract score. In another example, treatment induces ≥1 point decrease in liver score. In another example, treatment induces ≥1 point decrease in lung symptom score. In another example, treatment induces ≥1 point decrease in lung FEV1 score. In another example, treatment induces ≥1 point decrease in joints and fascia score. In another example, treatment induces ≥1 point decrease in genital tract score.

In an example, the treatment induces a complete response after treatment is initiated. In an example, a complete response is the complete resolution of GvHD symptoms in all organs. In an example, the complete response is induced 28 days after treatment is initiated. In an example, the complete response is induced at least 28 after treatment is initiated. In an example, the complete response is induced at least 30 after treatment is initiated. In an example, the complete response is induced at least 2 months after treatment is initiated. In another example, the complete response is induced at least 3 months after treatment is initiated. In another example, the complete response is induced 28 to 56 days after treatment is initiated. In another example, the complete response is induced 100 days after treatment is initiated. In another example, the complete response is induced 160 days after treatment is initiated. In another example, the complete response is induced 180 days after treatment is initiated.

In another example, the complete response is induced after two doses. In another example, the complete response is induced after administering doses twice weekly for four weeks. In another example, the complete response is induced after three doses or more.

In another example, the treatment increases the probability of the subject's survival. For example, treatment increases the probability of the subject surviving for at least 20 days to 200 days after initiation of treatment. In one example, treatment increases the probability of the subject surviving for at least 180 days after initiation of treatment. In another example, treatment increases the probability of the subject surviving at least 100 days. In an example, the increased probability is determined relative to a subject that is not treated with a composition of the disclosure.

In another example, treatment decreases the subject's risk of 6 month non-relapse mortality. For example, the subject's risk of 6 month non-relapse mortality decreases to between 20% and 80%. In one example, the subject's risk is decreased to at least 70%. In another example, the subject's risk is decreased to at least 60%. In another example, the subject's risk is decreased to at least 50%. In another example, the subject's risk is decreased to at least 40%. In another example, the subject's risk is decreased to at least 30%.

In another example, the treatment decreases the subject's risk of 1 year non-relapse mortality. For example, the subject's risk of 1 year non-relapse mortality is decreased to between 20% and 80%. In one example, the subject's risk is decreased to at least 70%. In another example, the subject's risk is decreased to at least 60%. In another example, the subject's risk is decreased to at least 50%. In another example, the subject's risk is decreased to at least 40%. In another example, the subject's risk is decreased to at least 30%.

In an example, treatment provides an overall response rate>40%. In an example, treatment provides an overall response rate>50%.

### Mesenchymal Lineage Precursor or Stem Cells

As used herein, the term “mesenchymal lineage precursor or stem cell (MLPSC)” refers to undifferentiated multipotent cells that have the capacity to self-renew while maintaining multipotency and the capacity to differentiate into a number of cell types either of mesenchymal origin, for example, osteoblasts, chondrocytes, adipocytes, stromal cells, fibroblasts and tendons, or non-mesodermal origin, for example, hepatocytes, neural cells and epithelial cells. For the avoidance of doubt, a “mesenchymal lineage precursor cell” refers to a cell which can differentiate into a mesenchymal cell such as bone, cartilage, muscle and fat cells, and fibrous connective tissue.

The term “mesenchymal lineage precursor or stem cells” includes both parent cells and their undifferentiated progeny. The term also includes mesenchymal precursor cells (MPCs), multipotent stromal cells, mesenchymal stem cells (MSCs), perivascular mesenchymal precursor cells, and their undifferentiated progeny.

MLPSCs can be autologous, allogeneic, xenogenic, syngenic or isogenic. Autologous cells are isolated from the same individual to which they will be reimplanted. Allogeneic cells are isolated from a donor of the same species. Xenogenic cells are isolated from a donor of another species. Syngenic or isogenic cells are isolated from genetically identical organisms, such as twins, clones, or highly inbred research animal models.

In an example, the MLPSCs are allogeneic. In an example, the allogeneic MLPSCs are culture expanded and cryopreserved.

MLPSCs reside primarily in the bone marrow, but have also shown to be present in diverse host tissues including, for example, cord blood and umbilical cord, adult peripheral blood, adipose tissue, trabecular bone and dental pulp. They are also found in skin, spleen, pancreas, brain, kidney, liver, heart, retina, brain, hair follicles, intestine, lung, lymph node, thymus, ligament, tendon, skeletal muscle, dermis, and periosteum; and are capable of differentiating into germ lines such as mesoderm and/or endoderm and/or ectoderm. Thus, MLPSCs are capable of differentiating into a large number of cell types including, but not limited to, adipose, osseous, cartilaginous, elastic, muscular, and fibrous connective tissues. The specific lineage-commitment and differentiation pathway which these cells enter depends upon various influences from mechanical influences and/or endogenous bioactive factors, such as growth factors, cytokines, and/or local microenvironmental conditions established by host tissues.

The terms “enriched”, “enrichment” or variations thereof are used herein to describe a population of cells in which the proportion of one particular cell type or the proportion of a number of particular cell types is increased when compared with an untreated population of the cells (e.g., cells in their native environment). In one example, a population enriched for MLPSCs comprises at least about 0.1% or 0.5% or 1% or 2% or 5% or 10% or 15% or 20% or 25% or 30% or 50% or 75% MLPSCs. In this regard, the term “population of cells enriched for MLPSCs” will be taken to provide explicit support for the term “population of cells comprising X % MLPSCs”, wherein X % is a percentage as recited herein. The MLPSCs can, in some examples, form clonogenic colonies, e.g. CFU-F (fibroblasts) or a subset thereof (e.g., 50% or 60% or 70% or 70% or 90% or 95%) can have this activity.

In an example, MLPSCs of the disclosure are culture expanded from a population of MLPSCs that are STRO-1+. In an example, the MLPSCs are culture expanded from a population of MLPSCs which comprise about 0.1% to 75% STRO-1+ cells. In another example, the MLPSCs are culture expanded from a population of MLPSCs which comprise about 0.5% to 75% STRO-1+ cells. In another example, the MLPSCs are culture expanded from a population of MLPSCs which comprise about 0.1% to 75% STRO-1+ cells. In another example, the MLPSCs are culture expanded from a population of MLPSCs which comprise about 0.1% to 75% STRO-1+ cells. In another example, the MLPSCs are culture expanded from a population of MLPSCs which comprise about 1% to 75% STRO-1+ cells. In another example, the MLPSCs are culture expanded from a population of MLPSCs which comprise about 0.1% to 75% STRO-1+ cells. In another example, the MLPSCs are culture expanded from a population of MLPSCs which comprise about 10% to 75% STRO-1+ cells.

In an example of the present disclosure, the MLPSCs are mesenchymal stem cells (MSCs). The MSCs may be a homogeneous composition or may be a mixed cell population enriched in MSCs. Homogeneous MSC compositions may be obtained by culturing adherent marrow or periosteal cells, and the MSCs may be identified by specific cell surface markers which are identified with unique monoclonal antibodies. A method for obtaining a cell population enriched in MSCs is described, for example, in U.S. Pat. No. 5,486,359. Alternative sources for MSCs include, but are not limited to, blood, skin, cord blood, muscle, fat, bone, and perichondrium. In an example, the MSCs are allogeneic. In an example, the MSCs are cryopreserved. In an example, the MSCs are culture expanded and cryopreserved.

In another example, the MLPSCs are CD29+, CD54+, CD73+, CD90+, CD102+, CD105+, CD106+, CD166+, MHC1+ MSCs.

Isolated or enriched MLPSCs can be expanded in vitro by culture. Isolated or enriched MLPSCs can be cryopreserved, thawed and subsequently expanded in vitro by culture.

In one example, isolated or enriched MLPSCs are seeded at 50,000 viable cells/cm² in culture medium (serum free or serum-supplemented), for example, alpha minimum essential media (αMEM) supplemented with 5% fetal bovine serum (FBS) and glutamine, and allowed to adhere to the culture vessel overnight at 37 °C, 20% O₂. The culture medium is subsequently replaced and/or altered as required and the cells cultured for a further 68 to 72 hours at 37 °C, 5% O₂.

As will be appreciated by those of skill in the art, cultured MLPSCs are phenotypically different to cells in vivo. For example, in one embodiment they express one or more of the following markers, CD44, NG2, DC146 and CD140b. Cultured MLPSCs are also biologically different to cells in vivo, having a higher rate of proliferation compared to the largely non-cycling (quiescent) cells in vivo.

In one example, the population of cells is enriched from a cell preparation comprising STRO-1+ cells in a selectable form. In this regard, the term “selectable form” will be understood to mean that the cells express a marker (e.g., a cell surface marker) permitting selection of the STRO-1+ cells. The marker can be STRO-1, but need not be. For example, as described and/or exemplified herein, cells (e.g., mesenchymal precursor cells (MPCs)) expressing STRO-2 and/or STRO-3 (TNAP) and/or STRO-4 and/or VCAM-1 and/or CD146 and/or 3G5 also express STRO-1 (and can be STRO-1bright). Accordingly, an indication that cells are STRO-1+ does not mean that the cells are selected solely by STRO-1 expression. In one example, the cells are selected based on at least STRO-3 expression, e.g., they are STRO-3+ (TNAP+). For example, the MPCs can be isolated from bone mononuclear cells with an anti-STRO-3 antibody.

Reference to selection of a cell or population thereof does not necessarily require selection from a specific tissue source. As described herein STRO-1+ cells can be selected from or isolated from or enriched from a large variety of sources. That said, in some examples, these terms provide support for selection from any tissue comprising STRO-1+ cells (e.g., mesenchymal precursor cells) or vascularized tissue or tissue comprising pericytes (e.g., STRO-1+ pericytes) or any one or more of the tissues recited herein.

In one example, the cells used in the present disclosure express one or more markers individually or collectively selected from the group consisting of TNAP+, VCAM-1+, THY-1+, STRO-2+, STRO-4+ (HSP-90β), CD45+, CD146+, 3G5+ or any combination thereof.

By “individually” is meant that the disclosure encompasses the recited markers or groups of markers separately, and that, notwithstanding that individual markers or groups of markers may not be separately listed herein the accompanying claims may define such marker or groups of markers separately and divisibly from each other.

By “collectively” is meant that the disclosure encompasses any number or combination of the recited markers or groups of markers, and that, notwithstanding that such numbers or combinations of markers or groups of markers may not be specifically listed herein the accompanying claims may define such combinations or sub-combinations separately and divisibly from any other combination of markers or groups of markers.

As used herein the term “TNAP” is intended to encompass all isoforms of tissue non-specific alkaline phosphatase. For example, the term encompasses the liver isoform (LAP), the bone isoform (BAP) and the kidney isoform (KAP). In one example, the TNAP is BAP. In one example, TNAP as used herein refers to a molecule which can bind the STRO-3 antibody produced by the hybridoma cell line deposited with ATCC on 19 Dec. 2005 under the provisions of the Budapest Treaty under deposit accession number PTA-7282.

Furthermore, in one example, the STRO-1+ cells are capable of giving rise to clonogenic CFU-F.

In one example, a significant proportion of the STRO-1+ cells are capable of differentiation into at least two different germ lines. Non-limiting examples of the lineages to which the STRO-1+ cells may be committed include bone precursor cells; hepatocyte progenitors, which are multipotent for bile duct epithelial cells and hepatocytes; neural restricted cells, which can generate glial cell precursors that progress to oligodendrocytes and astrocytes; neuronal precursors that progress to neurons; precursors for cardiac muscle and cardiomyocytes, glucose-responsive insulin secreting pancreatic beta cell lines. Other lineages include, but are not limited to, odontoblasts, dentin-producing cells and chondrocytes, and precursor cells of the following: retinal pigment epithelial cells, fibroblasts, skin cells such as keratinocytes, dendritic cells, hair follicle cells, renal duct epithelial cells, smooth and skeletal muscle cells, testicular progenitors, vascular endothelial cells, tendon, ligament, cartilage, adipocyte, fibroblast, marrow stroma, cardiac muscle, smooth muscle, skeletal muscle, pericyte, vascular, epithelial, glial, neuronal, astrocyte and oligodendrocyte cells.

In an example, MLPSCs are obtained from a single donor, or multiple donors where the donor samples or MLPSCs are subsequently pooled and then culture expanded.

MLPSCs encompassed by the present disclosure may also be cryopreserved prior to administration to a subject. In an example, MLPSCs are culture expanded and cryopreserved prior to administration to a subject.

In an example, the present disclosure encompasses MLPSCs as well as progeny thereof, soluble factors derived therefrom, and/or extracellular vesicles isolated therefrom. In another example, the present disclosure encompasses MLPSCs as well as extracellular vesicles isolated therefrom. For example, it is possible to culture expand MLPSCs of the disclosure for a period of time and under conditions suitable for secretion of extracellular vesicles into the cell culture medium. Secreted extracellular vesicles can subsequently be obtained from the culture medium for use in therapy.

The term “extracellular vesicles” as used herein, refers to lipid particles naturally released from cells and ranging in size from about 30 nm to as a large as 10 microns, although typically they are less than 200 nm in size. They can contain proteins, nucleic acids, lipids, metabolites, or organelles from the releasing cells (e.g., mesenchymal stem cells; STRO-1+ cells).

The term “exosomes” as used herein, refers to a type of extracellular vesicle generally ranging in size from about 30 nm to about 150 nm and originating in the endosomal compartment of mammalian cells from which they are trafficked to the cell membrane and released. They may contain nucleic acids (e.g., RNA; microRNAs), proteins, lipids, and metabolites and function in intercellular communication by being secreted from one cell and taken up by other cells to deliver their cargo.

### Culture Expansion

“Culture expanded” MLPSCs are distinguished from freshly isolated cells in that they have been cultured in cell culture medium and passaged (i.e. sub-cultured).

In an example, freshly isolated cells are culture expanded for about 1 or 2 passages to provide an intermediate population. In an example, freshly isolated cells are culture expanded for 2 passages to provide an intermediate population. In another example, freshly isolated cells are culture expanded for about 1 to 3 passages to provide an intermediate population. In an example, freshly isolated cells are STRO-1+. For example, the population of MLPSCs that are STRO-1+ can comprise about 0.1% to 75% STRO-1+ cells.

In an example, relevant cells are isolated and culture expanded for 2 passages to provide an intermediate MLPSC population. The intermediate MLPSC population is then culture expanded to provide a drug product (DP). For example, DP compositions of the present disclosure are produced by culturing cells from an intermediate cryopreserved MLPSC population or, put another way, a cryopreserved intermediate. In an example, the intermediate cell population can be cultured for three more passages (i.e. 5 passages total) to provide a DP.

In an example, MLPSCs are culture expanded for about 4-10 passages. In an example, MLPSCs are culture expanded for at least 5, at least 6, at least 7, at least 8, at least 9, at least 10 passages. For example, MLPSCs can be culture expanded for at least 5 passages. In an example, MLPSCs can be culture expanded for at least 5-10 passages. In an example, MLPSCs can be culture expanded for at least 5-8 passages. In an example, MLPSCs can be culture expanded for at least 5-7 passages. In an example, MLPSCs can be culture expanded for more than 7 passages. In these examples, MLPSCs may be culture expanded before being cryopreserved to provide an intermediate cryopreserved MLPSC population and then subject to further culture expansion.

In an example, compositions of the disclosure comprise MLPSCs that are culture expanded from a cryopreserved intermediate. In an example, the cells culture expanded from a cryopreserved intermediate are culture expanded for at least 3, at least 5, at least 6, at least 7, at least 8, at least 9, at least 10 passages. For example, MLPSCs can be culture expanded for at least 3 passages. In an example, MLPSCs can be culture expanded for at least 3-10 passages. In an example, MLPSCs can be culture expanded for at least 3-8 passages. In an example, MLPSCs can be culture expanded for at least 3-7 passages.

In an example, MLPSCs can be obtained from a single donor, or multiple donors where the donor samples or MLPSCs are subsequently pooled and then culture expanded as required. In an example, the culture expansion process comprises:
i. expanding by passage expansion the number of viable cells to provide a preparation of at least about 1 billion of the viable cells, wherein the passage expansion comprises establishing a primary culture of isolated MLPSCs and then serially establishing a first non-primary (P1) culture of isolated MLPSCs from the previous culture;
ii. expanding by passage expansion the P1 culture of isolated MLPSCs to a second non-primary (P2) culture of MLPSCs; and,
iii. preparing and cryopreserving an in-process intermediate MLPSC preparation obtained from the P2 culture of MLPSCs; and, optionally
iv. thawing the cryopreserved in-process intermediate MLPSC preparation and expanding by passage expansion the in-process intermediate MLPSC preparation.

In an example, the expanded MLPSC preparation has an profile comprising:
i. less than about 0.75% CD45+ cells;
ii. at least about 95% CD105+ cells;
iii. at least about 95% CD166+ cells.

The process of MLPSC isolation and ex vivo expansion can be performed using any equipment and cell handing methods known in the art. Various culture expansion embodiments of the present disclosure employ steps that require manipulation of cells, for example, steps of seeding, feeding, dissociating an adherent culture, or washing. Any step of manipulating cells has the potential to insult the cells. Although MLPSCs can generally withstand a certain amount of insult during preparation, cells are preferably manipulated by handling procedures and/or equipment that adequately performs the given step(s) while minimizing insult to the cells.

In an example, MLPSCs are washed in an apparatus that includes a cell source bag, a wash solution bag, a recirculation wash bag, a spinning membrane filter having inlet and outlet ports, a filtrate bag, a mixing zone, an end product bag for the washed cells, and appropriate tubing, for example, as described in U.S. Pat. No. 6,251,295, which is hereby incorporated by reference.

In an example, a MLPSC composition cultured according to the present disclosure is 95% homogeneous with respect to being CD105 positive and CD166 positive and being CD45 negative. In an example, this homogeneity persists through ex vivo expansion; i.e. though multiple population doublings.

In an example, MLPSCs of the disclosure are culture expanded in 2D culture. For example, MLPSCs of the disclosure can be culture expanded in a cell factory. In certain examples, 3D culture of intermediates disclosed herein may follow using, for example, a bioreactor. In an example, MLPSCs of the disclosure are initially culture expanded in 2D culture prior to being further expanded in 3D culture. In an example, intermediate cell populations of the disclosure have not been culture expanded in 3D culture.

In an example, MLPSCs of the disclosure are culture expanded from an intermediate population. In an example, MLPSCs of the disclosure are culture expanded from the intermediate in 2D culture before seeding in 3D culture.

In the context of both intermediate populations and therapeutic compositions expanded from the same, in an example, MLPSCs of the disclosure are culture expanded in 2D culture for at least 3 days before seeding in a further culture system such as cell factory or 3D culture in a bioreactor. In an example, MLPSCs of the disclosure are culture expanded in 2D culture for at least 4 days before seeding in a further culture system. In an example, MLPSCs of the disclosure are culture expanded in 2D culture for between 3 and 5 days before seeding in a further culture system. In these examples, 2D culture can be performed in a cell factory. Various cell factory products are available commercially (e.g. Thermofisher, Sigma, Corning). In an example, the cell factory has at least 5 layers. In an example, the cell factory has at least 10 layers. In an example, the cell factory has at least 20 layers. 3D culture may be performed in various bioreactor types such as stirred tank, wave bag, and vertical wheel.

In an example, CO₂ is provided during culture expansion of MLPSCs. In an example, MLPSCs are culture expanded in less than 9% CO₂. In an example, MLPSCs are culture expanded in less than 8% CO₂. In an example, MLPSCs are culture expanded in 5% CO₂. For example, MLPSCs can be culture expanded in 5%+/−2% CO₂. In an example, the MLPSCs are culture expanded with passive priming of CO₂. For example, cell factories can be passively primed with 5% CO₂.

Priming cell factories maintains the CO₂ tension between the cell factory and incubator and stabilizes the pH level of the growth medium. Active priming involves actively passing CO₂ gas through a bacterial vent air filter into each culture vessel (e.g. cell factory) for a defined period of time (e.g. around 10 minutes). However, active priming has the potential to introduce contamination into culture as it requires an open port to provide gas. Passive priming involves placing a closed culture system into an incubator at appropriate CO₂ concentration prior to cell seeding (e.g. around 12 to 72 hours).

In an example, cells of the disclosure are STRO-3+ before they are culture expanded to provide an intermediate cell population.

### Cell Culture Medium

MLPSCs disclosed herein can be culture expanded in various suitable growth mediums.

The term “medium” or “media” as used in the context of the present disclosure, includes the components of the environment surrounding the cells. The media contributes to and/or provides the conditions suitable to allow cells to grow. Media may be solid, liquid, gaseous or a mixture of phases and materials. Media can include liquid growth media as well as liquid media that do not sustain cell growth. Media also include gelatinous media such as agar, agarose, gelatin and collagen matrices. Exemplary gaseous media include the gaseous phase that cells growing on a petri dish or other solid or semisolid support are exposed to.

The cell culture media used for culture expansion contains all essential amino acids and may also contain non-essential amino acids. In general, amino acids are classified into essential amino acids (Thr, Met, Val, Leu, Ile, Phe, Trp, Lys, His) and non-essential amino acids (Gly, Ala, Ser, Cys, Gln, Asn, Asp, Tyr, Arg, Pro).

Those of skill in the art will appreciate that for optimal results, the basal medium must be appropriate for the cell line of interest. For example, it may be necessary to increase the level of glucose (or other energy source) in the basal medium, or to add glucose (or other energy source) during the course of culture, if this energy source is found to be depleted and to thus limit growth. In an example, dissolved oxygen (DO) levels can also be controlled.

In an example, the cell culture medium contains human derived additives. For example, human serum and human platelet cell lysate can be added to the cell culture media.

In an example, the cell culture medium contains only human derived additives. Thus, in an example, the cell culture media is xeno-free. For avoidance of doubt, in these examples, the culture medium is free of animal proteins. In an example, cell culture medium used in the methods of the disclosure is free of animal components.

In an example, the culture medium comprises serum. In other examples the culture medium is fetal bovine serum free culture medium comprising growth factors that promote MLPSCs proliferation. In an embodiment, the culture medium is serum free stem cell culture medium. In an example, the cell culture medium comprises:
a basal medium;
platelet derived growth factor (PDGF);
fibroblast growth factor 2 (FGF2).

In an example, the culture medium comprises platelet derived growth factor (PDGF) and fibroblast growth factor 2 (FGF2), wherein the level of FGF2 is less than about 6 ng/ml. For example, the FGF2 level may be less than about 5 ng/ml, less than about 4 ng/ml, less than about 3 ng/ml, less than about 2 ng/ml, less than about 1 ng/ml. In other examples, the FGF2 level is less than about 0.9 ng/ml, less than about 0.8 ng/ml, less than about 0.7 ng/ml, less than about 0.6 ng/ml, less than about 0.5 ng/ml, less than about 0.4 ng/ml, less than about 0.3 ng/ml, less than about 0.2 ng/ml.

In another example, the level of FGF2 is between about 1 μg/ml and 100 μg/ml. In another example, the level of FGF2 is between about 5 μg/ml and 80 μg/ml. In an example, the level of FGF2 is about 1 ng/ml.

In an example, the PDGF is PDGF-BB. In an example, the level of PDGF-BB is between about 1 ng/ml and 150 ng/ml. In another example, the level of PDGF-BB is between about 7.5 ng/ml and 120 ng/ml. In another example, the level of PDGF-BB is between about 15 ng/ml and 60 ng/ml. In another example, the level of PDGF-BB is at least about 10 ng/ml. In another example, the level of PDGF-BB is at least about 15 ng/ml. In another example, the level of PDGF-BB is at least about 20 ng/ml. In another example, the level of PDGF-BB is at least about 21 ng/ml. In another example, the level of PDGF-BB is at least about 22 ng/ml. In another example, the level of PDGF-BB is at least about 23 ng/ml. In another example, the level of PDGF-BB is at least about 24 ng/ml. In another example, the level of PDGF-BB is at least about 25 ng/ml.

In another example, the PDGF is PDGF-AB. In an example, the level of PDGF-AB is between about 1 ng/ml and 150 ng/ml. In another example, the level of PDGF-AB is between about 7.5 ng/ml and 120 ng/ml. In another example, the level of PDGF-AB is between about 15 ng/ml and 60 ng/ml. In another example, the level of PDGF-AB is at least about 10 ng/ml. In another example, the level of PDGF-AB is at least about 15 ng/ml. In another example, the level of PDGF-AB is at least about 20 ng/ml. In another example, the level of PDGF-AB is at least about 21 ng/ml. In another example, the level of PDGF-AB is at least about 22 ng/ml. In another example, the level of PDGF-AB is at least about 23 ng/ml. In another example, the level of PDGF-AB is at least about 24 ng/ml. In another example, the level of PDGF-AB is at least about 25 ng/ml.

In other examples, additional factors can be added to the cell culture medium. In an example, the culture medium further comprising EGF. EGF is a growth factor that stimulates cell proliferation by binding to its receptor EGFR. In an example, the method of the present disclosure comprises culturing a population of stem cells in a fetal bovine serum free cell culture medium further comprising EGF. In an example, the level of EGF is between about 0.1 and 7 ng/ml. For example, the level of EGF can be at least about 5 ng/ml.

In another example, the level of EGF is between about 0.2 ng/ml and 3.2 ng/ml. In another example, the level of EGF is between about 0.4 ng/ml and 1.6 ng/ml. In another example, the level of EGF is between about 0.2 ng/ml. In another example, the level of EGF is at least about 0.3 ng/ml. In another example, the level of EGF is at least about 0.4 ng/ml. In another example, the level of EGF is at least about 0.5 ng/ml. In another example, the level of EGF is at least about 0.6 ng/ml. In another example, the level of EGF is at least about 0.7 ng/ml. In another example, the level of EGF is at least about 0.8 ng/ml. In another example, the level of EGF is at least about 0.9 ng/ml. In another example, the level of EGF is at least about 1.0 ng/ml.

In the above examples, basal medium such as Alpha MEM or StemSpan™ can be supplemented with the referenced quantity of growth factor. In an example, the culture medium comprises Alpha MEM or StemSpan™ supplemented with 32 ng/ml PDGF-BB, 0.8 ng/ml EGF and 0.02 ng/ml FGF2. In an example, the culture medium comprises Alpha MEM or StemSpan™ supplemented with 10 ng/ml PDGF-BB, 5 ng/ml EGF and 1 ng/ml FGF2.

In other examples, additional factors can be added to the cell culture medium. For example, the cell culture media can be supplemented with one or more stimulatory factors selected from the group consisting of epidermal growth factor (EGF), 1α,25-dihydroxyvitamin D3 (1,25D), tumor necrosis factor α (TNF-α), interleukin-1β (IL-1β) and stromal derived factor 1α (SDF-1α). In another embodiment, cells may also be cultured in the presence of at least one cytokine in an amount adequate to support growth of the cells. In another embodiment, cells can be cultured in the presence of heparin or a derivative thereof. For example, the cell culture medium may contain about 50 ng/ml of heparin. In other examples, the cell culture medium contains about 60 ng/ml of heparin, about 70 ng/ml of heparin, about 80 ng/ml of heparin, about 90 ng/ml of heparin, about 100 ng/ml of heparin, about 110 ng/ml of heparin, about 110 ng/ml of heparin, about 120 ng/ml of heparin, about 130 ng/ml of heparin, about 140 ng/ml of heparin, about 150 ng/ml of heparin or a derivative thereof. In an example, the heparin derivative is a sulphate). Various forms of heparin sulphate are known in the art and include heparin sulphate 2 (HS2). HS2 can be derived from various sources including for example, the liver of male and/or female mammals. Thus, an exemplary heparin sulphate includes male liver heparin sulphate (MML HS) and female liver heparin sulphate (FML HS).

In another example, the cell culture medium of the present disclosure promotes stem cell proliferation while maintaining stem cells in an undifferentiated state. Stem cells are considered to be undifferentiated when they have not committed to a specific differentiation lineage. As discussed above, stem cells display morphological characteristics that distinguish them from differentiated cells. Furthermore, undifferentiated stem cells express genes that may be used as markers to detect differentiation status. The polypeptide products may also be used as markers to detect differentiation status. Accordingly, one of skill in the art could readily determine whether the methods of the present disclosure maintain stem cells in an undifferentiated state using routine morphological, genetic and/or proteomic analysis.

### Media Supplement(s)

In certain examples, MLPSCs of the disclosure are culture expanded in media supplemented with pro-inflammatory cytokines and/or non-fetal serum. In an example, the cell culture media comprises IFN-gamma and/or TNF-alpha. In an example, the cell culture media comprises IFN-gamma. For example, the level of IFN-gamma can be less than 1 ng/ml. In an example the level of IFN-gamma is less than 500 pg/ml or less than 100 pg/ml. In an example, the cell culture media comprises TNF-alpha. For example, the level of TNF-alpha can be less than 1 ng/ml. In an example, the level of TNF-alpha is less than 750 pg/ml or less than 400 pg/ml. In an example, the cell culture media comprises IFN-gamma and TNF-alpha and the level of both is less than 1 ng/ml.

In an example, the cell culture media comprises one or more pro-inflammatory cytokines which are capable of binding a receptor on the surface of MLPSCs.

In an example, the cell culture media comprises one or more pro-inflammatory cytokines selected from the group consisting of IL-6; IL-8; IL-17A; MCP-1; MIP-1-alpha; MIP-1-beta; IP-10. For example, the cell culture media can comprise IL-8.

In an example, the cell culture media comprises IFN-gamma and/or TNF-alpha, and, one or more pro-inflammatory cytokines selected from the group consisting of IL-6; IL-8; IL-17A; MCP-1; MIP-1-alpha; MIP-1-beta; IP-10. In an example, the level of IFN-gamma and/or TNF-alpha is less than 1 ng/ml.

In an example, the cell culture media is characterised by one or more or all of the following:
i. a level of IFN-gamma greater than 1 μg/ml;
ii. a level of TNF-alpha greater than 2 μg/ml;
iii. a level of IL-6 greater than 3 μg/ml;
iv. a level of IL-8 greater than 500 μg/ml;
v. a level of IL-17A greater than 0.2 μg/ml; V.
vi. a level of MCP-1 greater than 3 μg/ml;
vii. a level of MIP-1-alpha greater than 0.5 μg/ml;
viii. a level of MIP-1-beta greater than 3 μg/ml;
ix. a level of IP-10 greater than 500 μg/ml.

In another example, the media comprises serum characterised by one or more or all of the following:
i. a level of IFN-gamma greater than 10 μg/ml;
ii. a level of TNF-alpha greater than 20 μg/ml;
iii. a level of IL-6 greater than 30 μg/ml;
iv. a level of IL-8 greater than 5,000 μg/ml;
v. a level of IL-17A greater than 2 μg/ml;
vi. a level of MCP-1 greater than 30 μg/ml;
vii. a level of MIP-1-alpha greater than 50 μg/ml;
viii. a level of MIP-1-beta greater than 30 μg/ml;
ix. a level of IP-10 greater than 5,000 μg/ml.

In an example, the media comprises IL-10. In another example, the media comprises IL-36RA. In another example, the media comprises IL-10 and IL-36RA. In an example, the level of IL-10 is greater than 0.3 pg/ml. For example, the level of IL-10 may be greater than 30 pg/ml. In an example, the level of IL-10 is greater than 400 pg/ml. In an example, the level of IL-36RA is greater than 50 pg/ml.

In another example, methods of the disclosure encompass culture expansion in cell culture media which comprise newborn serum. Various examples of suitable serum (and levels of the same) are disclosed herein.

### Serum

“Newborn serum” refers to serum that has been obtained postpartum. For example, the culture media can be supplemented with mammalian newborn serum (e.g. bovine). In an example, the culture media can be supplemented with animal newborn serum. In another example, the culture media can be supplemented with human newborn serum.

In an example, the cell culture media is supplemented with at least about 1% v/v, at least about 2% v/v, at least about 3% v/v, at least about 4% v/v, at least about 5% v/v, at least about 6% v/v, at least about 7% v/v, at least about 8% v/v, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25% v/v newborn serum. In an example, the cell culture media is supplemented with between about 1% v/v and about 15% v/v newborn serum. In an example, the cell culture media is supplemented with between about 1% v/v and about 10% v/v newborn serum. In an example, the cell culture media is supplemented with between about 5% v/v and about 10% v/v newborn serum. In an example, the cell culture media is supplemented with about 5% v/v newborn serum.

In an example, the newborn serum comprises at least one inflammatory cytokine. As used herein, the term “inflammatory cytokine” refers to a signalling molecule that promotes inflammation. In example, the one or more cytokine is selected from the group comprising IL-1β, IL-6, TNF-α, IFN-γ and/or IL-1ra.

In an example, the newborn serum comprises IFN-gamma. In another example, the newborn serum comprises TNF-alpha. In another example, the newborn serum comprises IFN-gamma and TNF-alpha. In another example, the newborn serum comprises one or more pro-inflammatory cytokines selected from the group consisting of IL-6; IL-8; IL-17A; MCP-1; MIP-1-alpha; MIP-1-beta; IP-10. For example, the newborn serum can comprise IL-8. In an example, the newborn serum comprises IFN-gamma and/or TNF-alpha and, one or more pro-inflammatory cytokines selected from the group consisting of IL-6; IL-8; IL-17A; MCP-1; MIP-1-alpha; MIP-1-beta; IP-10. In another example, the newborn serum comprises IFN-gamma and TNF-alpha and, one or more pro-inflammatory cytokines selected from the group consisting of IL-6; IL-8; IL-17A; MCP-1; MIP-1-alpha; MIP-1-beta; IP-10. In an example, the level of IFN-gamma is less than 1 ng/ml. In an example, the level of TNF-alpha is less than 1 ng/ml. In an example, the level of both IFN-gamma and TNF-alpha are less than 1 ng/ml. For example, the level of IFN-gamma may be less than 500 pg/ml or less than 100 pg/ml. In an example, the level of TNF-alpha is less than 750 pg/ml or less than 400 pg/ml.

Methods to detect the presence of cytokines in serum are known in the art and include, for example, enzyme-linked immunosorbent assay (ELISA). In another example, the presence of cytokines in serum are detected by measuring cytokine mRNA, for example by polymerase-chain reaction (PCR) techniques such as reverse-transcription PCR.

In an example, the newborn serum can be newborn calf serum (NBCS). In an example, the NBCS is obtained from newborn calves who have been fed colostrum. In an example, NBCS comprises elevated levels of at least one inflammatory cytokine relative to NBCS obtained from a calf that has not been fed colostrum. In an example, NBCS comprises elevated levels of at least one inflammatory cytokine relative to fetal serum such as FCS.

In an example, the NBCS is obtained within 4 weeks after birth of the calf. In an example, the NBCS is obtained within 21 days after birth of the calf. For example, the NBCS is obtained≤21 days after birth of the calf. In an example, the NBCS is obtained between the day of birth and 21 days after birth of the calf. In an example, the NBCS is obtained between the day of birth and 14 days after birth of the calf. In an example, the NBCS is obtained between the day of birth and 10 days after birth of the calf. In an example, the NBCS is obtained between the day of birth and 7 days after birth of the calf. In an example, the NBCS is obtained between 6 hours after birth and 72 hours after birth. In an example, the NBCS is obtained between 6 hours after birth and 48 hours after birth. In an example, the NBCS is obtained between 6 hours after birth and 24 hours after birth. In an example, the NBCS is obtained between 12 hours after birth and 24 hours after birth.

In an example, the cell culture media is supplemented with at least about 1% v/v, at least about 2% v/v, at least about 3% v/v, at least about 4% v/v, at least about 5% v/v, at least about 6% v/v, at least about 7% v/v, at least about 8% v/v, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25% v/v NBCS. In an example, the cell culture media is supplemented with between about 1% v/v and about 15% v/v NBCS. In an example, the cell culture media is supplemented with between about 5% v/v and about 10% v/v NBCS. In an example, the cell culture media is supplemented with at least about 5% v/v NBCS.

In an example, the culture medium is also supplemented with fetal serum. In an example, the fetal serum is fetal calf serum (FCS). It is envisaged that the term fetal calf serum (FCS) and fetal bovine serum (FBS) can in the context of the present disclosure be used interchangeably. In an example, cell culture medium is supplemented with less than 10% v/v FCS. In an example, cell culture medium is supplemented with about 5% v/v FCS.

In an example, the cell culture medium is fetal serum free.

In an example, the cell culture medium is FCS free.

In an example, the culture media is supplemented with a mixture of FCS and NBCS. In an example the cell culture medium is supplemented with about 5% v/v FCS and about 5% v/v NBCS (i.e. a 1:1 ratio of FCS to NBCS). In an example, the culture media can be supplemented with a mixture of FCS and NBCS so that the FCS:NBCS ratio is at least about 0.4:1, at least about 0.5:1, at least about 0.6:1, at least about 0.7:1, at least about 0.8:1, at least about 0.9:1, at least about 1:1, at least about 1.5:1, at least about 2:1. In an example, the FCS:NBCS ratio is between about 0.5:1 and about 2:1. In an example, the FCS:NBCS ratio is between about 0.8:1 and about 1.5:1. In an example, the FCS:NBCS ratio is between about 0.8:1 and about 1.2:1. In an example, the FCS:NBCS ratio is about 1:1.

In an example, the mixture of FCS and NBCS can comprise at least about 1% v/v, at least about 2% v/v, at least about 3% v/v, at least about 4% v/v, at least about 5% v/v, at least about 6% v/v, at least about 7% v/v, at least about 8% v/v, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25% v/v of the cell culture media. In an example, the mixture of FCS and NBCS can comprise between about 1% v/v and about 15% v/v of the cell culture media. In an example, the mixture of FCS and NBCS can comprise between about 2% v/v and about 12% v/v of the cell culture media. In an example, the mixture of FCS and NBCS can comprise between about 5% v/v and about 12% v/v of the cell culture media. In an example, the mixture of FCS and NBCS can comprise between about 8% v/v and about 12% v/v of the cell culture media. In an example, the mixture of FCS and NBCS can comprise about 10% v/v of the cell culture media However, in this example, the cell culture media is supplemented with at least about 1% v/v, at least about 2% v/v, at least about 3% v/v, at least about 4% v/v, at least about 5% v/v, at least about 6% v/v, at least about 7% v/v, at least about 8% v/v, at least about 9% v/v, but less than 10% v/v FCS. In an example, the cell culture media is supplemented with between about 1% v/v and about 9% v/v FCS. In an example, the cell culture media is supplemented with between about 3% v/v and about 8% v/v FCS. In an example, the cell culture media is supplemented with between about 3% v/v and about 6% v/v FCS. In an example, the cell culture media is supplemented with about 5% v/v FCS.

### Ascorbic Acid

In an example, the cell culture media is supplemented with a short acting ascorbic acid derivative. The term “short acting” encompasses ascorbic acid derivatives that are oxidised by approximately 80-90% following 24 hours of cell culture under culture conditions of neutral pH and 37 °C. In one example, the short acting L-ascorbic acid derivative is a L-ascorbic acid salt, for example L-ascorbic acid sodium salt. In an example, the cell culture media may contain at least about 0.005 g/L of a short acting ascorbic acid derivative. In another example, the cell culture media may contain at least about 0.01 g/L of a short acting ascorbic acid derivative. For example, the cell culture media may contain at least about 0.02 g/L of a short acting ascorbic acid derivative. In another example, the cell culture media may contain at least about 0.03 g/L of a short acting ascorbic acid derivative. For example, the cell culture media may contain at least about 0.04 g/L of a short acting ascorbic acid derivative. In another example, the cell culture media may contain at least about 0.05 g/L of a short acting ascorbic acid derivative. In another example, the cell culture media may contain at least about 0.06 g/L of a short acting ascorbic acid derivative.

In another example, the cell culture media contains a short acting ascorbic acid derivative but does not contain a substantial amount of a long-acting ascorbic acid derivative. For example, the cell culture media may contain a short acting ascorbic acid derivative but not more than 0.04 g/L of a long acting ascorbic acid derivative. In another example, the cell culture media may contain a short acting ascorbic acid derivative but not more than 0.03 g/L of a long-acting ascorbic acid derivative. In another example, the cell culture media may contain a short acting ascorbic acid derivative but not more than 0.02 g/L of a long-acting ascorbic acid derivative. In another example, the cell culture media may contain a short acting ascorbic acid derivative but not more than 0.01 g/L of a long-acting ascorbic acid derivative. In another example, the cell culture media may contain a short acting ascorbic acid derivative but not more than 0.005 g/L of a long-acting ascorbic acid derivative. In another example, the cell culture media may contain a short acting ascorbic acid derivative but not a long-acting ascorbic acid derivative. In another example, the cell culture media contains L-ascorbate sodium salt but does not contain a substantial amount of L-ascorbic acid-2-phosphate.

### Other Additives

In an example, the cell culture medium contains human derived additives. For example, human serum and human platelet cell lysate can be added to the cell culture media. In other examples, additional factors can be added to the cell culture medium. For example, the cell culture media can be supplemented with one or more stimulatory factors selected from the group consisting of, platelet derived growth factor (PDGF), fibroblast growth factor 2 (FGF2), epidermal growth factor (EGF), epidermal growth factor (EGF), 1α,25-dihydroxyvitamin D3 (1,25D), tumor necrosis factor α (TNF-α), interleukin-1β (IL-1β) and stromal derived factor 1α (SDF-1α). In another embodiment, cells may also be cultured in the presence of at least one cytokine in an amount adequate to support growth of the cells. In another embodiment, cells can be cultured in the presence of heparin or a derivative thereof.

In the above examples, basal medium such as Alpha MEM or StemSpan™ can be supplemented with the referenced quantity of serum and, in certain examples, other additives. Further examples of suitable culture mediums for culturing stem cells can be found, for example, in WO2016139340.

### Modification of the Cells

The MLPSCs disclosed herein may be altered in such a way that upon administration, lysis of the cell is inhibited. Alteration of an antigen can induce immunological non-responsiveness or tolerance, thereby preventing the induction of the effector phases of an immune response (e.g., cytotoxic T cell generation, antibody production etc.) which are ultimately responsible for rejection of foreign cells in a normal immune response. Antigens that can be altered to achieve this goal include, for example, MHC class I antigens, MHC class II antigens, LFA-3 and ICAM-1.

The MLPSCs may also be genetically modified to express proteins of importance for the differentiation and/or maintenance of striated skeletal muscle cells. Exemplary proteins include growth factors (TGF-β, insulin-like growth factor 1 (IGF-1), FGF), myogenic factors (e.g. myoD, myogenin, myogenic factor 5 (Myf5), myogenic regulatory factor (MRF)), transcription factors (e.g. GATA-4), cytokines (e.g. cardiotropin-1), members of the neuregulin family (e.g. neuregulin 1, 2 and 3) and homeobox genes (e.g. Csx, tinman and NKx family).

### Compositions

MLPSCs disclosed herein can be culture expanded from a cryopreserved intermediate to produce a preparation containing at least one therapeutic dose.

In an example, compositions of the disclosure comprise between 10×10⁶ cells and 35×10⁶ cells. In another example, the composition comprises between 20×10⁶ cells and 30×10⁶ cells. In other examples, the composition comprises at least 100×10⁶ cells. In another example, the composition comprises between 50×10⁶ cells and 500×10⁶ cells. In other examples, compositions of the disclosure comprise 150 million cells. In another example, compositions of the disclosure comprise between 20 million and 100 million cells. In an example compositions of the disclosure comprise between 6 and 7 million cells/ml in 3.8 ml.

In an example, compositions of the disclosure comprise conditioned media obtained from MLPSCs disclosed herein.

In one example, compositions of the disclose comprise a pharmaceutically acceptable carrier and/or excipient. The terms “carrier” and “excipient” refer to compositions of matter that are conventionally used in the art to facilitate the storage, administration, and/or the biological activity of an active compound (see, e.g., Remington's Pharmaceutical Sciences, 16th Ed., Mac Publishing Company (1980). A carrier may also reduce any undesirable side effects of the active compound. A suitable carrier is, for example, stable, e.g., incapable of reacting with other ingredients in the carrier. In one example, the carrier does not produce significant local or systemic adverse effect in recipients at the dosages and concentrations employed for treatment.

Suitable carriers for the present disclosure include those conventionally used, e.g., water, saline, aqueous dextrose, lactose, Ringer's solution, a buffered solution, hyaluronan and glycols are exemplary liquid carriers, particularly (when isotonic) for solutions. Suitable pharmaceutical carriers and excipients include starch, cellulose, glucose, lactose, sucrose, gelatin, malt, rice, flour, chalk, silica gel, magnesium stearate, sodium stearate, glycerol monostearate, sodium chloride, glycerol, propylene glycol, water, ethanol, and the like.

In another example, a carrier is a media composition, e.g., in which a cell is grown or suspended. Such a media composition does not induce any adverse effects in a subject to whom it is administered. Exemplary carriers and excipients do not adversely affect the viability of a cell and/or the ability of a cell to treat or prevent disease.

In one example, the carrier or excipient provides a buffering activity to maintain the cells and/or soluble factors at a suitable pH to thereby exert a biological activity, e.g., the carrier or excipient is phosphate buffered saline (PBS). PBS represents an attractive carrier or excipient because it interacts with cells and factors minimally and permits rapid release of the cells and factors, in such a case, the composition of the disclosure may be produced as a liquid for direct application to the blood stream or into a tissue or a region surrounding or adjacent to a tissue, e.g., by injection.

Compositions of the disclosure may be cryopreserved. Cryopreservation of mesenchymal lineage precursor or stem cells can be carried out using slow-rate cooling methods or ‘fast’ freezing protocols known in the art. Preferably, the method of cryopreservation maintains similar phenotypes, cell surface markers and growth rates of cryopreserved cells in comparison with unfrozen cells.

The cryopreserved composition may comprise a cryopreservation solution. The pH of the cryopreservation solution is typically 6.5 to 8, preferably 7.4.

The cyropreservation solution may comprise a sterile, non-pyrogenic isotonic solution such as, for example, PlasmaLyte ATM. 100 mL of PlasmaLyte ATM contains 526 mg of sodium chloride, USP (NaCl); 502 mg of sodium gluconate (C6H11NaO7); 368 mg of sodium acetate trihydrate, USP (C2H3NaO2·3H2O); 37 mg of potassium chloride, USP (KCl); and 30 mg of magnesium chloride, USP (MgCl2·6H2O). It contains no antimicrobial agents. The pH is adjusted with sodium hydroxide. The pH is 7.4 (6.5 to 8.0).

The cryopreservation solution may comprise Profreeze™. The cryopreservation solution may additionally or alternatively comprise culture medium, for example, αMEM.

To facilitate freezing, a cryoprotectant such as, for example, dimethylsulfoxide (DMSO), is usually added to the cryopreservation solution. Ideally, the cryoprotectant should be nontoxic for cells and patients, nonantigenic, chemically inert, provide high survival rate after thawing and allow transplantation without washing. However, the most commonly used cryoprotector, DMSO, shows some cytotoxicity. Hydroxylethyl starch (HES) may be used as a substitute or in combination with DMSO to reduce cytotoxicity of the cryopreservation solution.

The cryopreservation solution may comprise one or more of DMSO, hydroxyethyl starch, human serum components and other protein bulking agents. In one example, the cryopreserved solution comprises Plasma-Lyte A (70%), DMSO (10%), HSA (25%) solution, the HSA solution comprising 5% HSA and 15% buffer.

In an example, the cryopreservation solution may further comprise one or more of methylcellulose, polyvinyl pyrrolidone (PVP) and trehalose.

The cryopreserved composition may be thawed and administered directly to the subject or added to another solution, for example, comprising hyaluronic acid. Alternatively, the cryopreserved composition may be thawed and the mesenchymal lineage precursor or stem cells resuspended in an alternate carrier prior to administration.

The compositions described herein may be administered alone or as admixtures with other cells. The cells of different types may be admixed with a composition of the disclosure immediately or shortly prior to administration, or they may be co-cultured together for a period of time prior to administration.

In one example, the composition comprises an effective amount or a therapeutically or prophylactically effective amount of mesenchymal lineage precursor or stem cells and/or progeny thereof and/or soluble factor derived therefrom. For example, the composition comprises about 1×10⁵ stem cells to about 1×10⁹ stem cells or about 1.25×10³ stem cells to about 1.25×10⁷ stem cells/kg (80 kg subject). The exact amount of cells to be administered is dependent upon a variety of factors, including the age, weight, and sex of the subject, and the extent and severity of the disorder being treated.

Despite the number of cells provided in the composition, in an example, between 1×10⁶ cells/kg and 5×10⁶ cells/kg are administered. In an example, 2×10⁶ cells/kg are administered. In an example, two doses of 2×10⁶ cells/kg are administered.

In an example, 50×10⁶ to 200×10⁷ cells are administered. In other examples, 60×10⁶ to 200×10⁶ cells or 75×10⁶ to 150×10⁶ cells are administered. In an example, 75×10⁶ cells are administered. In another example, 150×10⁶ cells are administered. In another example, between 1×10⁷ and 2×10⁸ cells are administered.

In an example, the composition comprises greater than 5.00×10⁶ viable cells/mL. In another example, the composition comprises greater than 5.50×10⁶ viable cells/mL. In another example, the composition comprises greater than 6.00×10⁶ viable cells/mL. In another example, the composition comprises greater than 6.50×10⁶ viable cells/mL. In another example, the composition comprises greater than 6.68×10⁶ viable cells/mL. In another example, the composition comprises 6.68×10⁶ cells/mL in 3.8 ml. In another example, the composition comprises 6.68×10⁶ viable cells/mL in 3.8 ml.

In an example, the mesenchymal lineage precursor or stem cells comprise at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99% of the cell population of the composition.

In an example, the culture expanded MLPSCs in the administered composition inhibit IL2-RA by at least 56% under culture conditions. In an example, the culture expanded MLPSCs in the administered composition inhibit IL2-RA by at least 60% under culture conditions. In an example, the culture expanded MLPSCs in the administered composition inhibit IL2-RA by at least 70% under culture conditions. In another example, the culture expanded MLPSCs in the administered composition inhibit IL2-RA by at least 75% under culture conditions. In another example, the culture expanded MLPSCs in the administered composition inhibit IL2-RA by between 55% and 75% under culture conditions. In another example, the culture expanded MLPSCs in the administered composition inhibit IL2-RA by between 55% and 75% under culture conditions. In another example, the culture expanded MLPSCs in the administered composition inhibit IL2-RA by between 60% and 75% under culture conditions.

In an example, the composition may optionally be packaged in a suitable container with written instructions for a desired purpose.

In an example, MLPSCs may be administered to a wall of a subject's gastrointestinal tract. In an example, MLPSCs can be administered to a site of inflammation in a subject's gastrointestinal tract wall. For example, MLPSCs can be administered into a site of inflammation in a subject's gastrointestinal tract wall. In these examples, the site of inflammation may be endoscopically confirmed prior to administration. For example, endoscopic confirmation can be based on visual inspection by a trained physician and/or histological analysis of endoscopic biopsy. In another example, compositions of the disclosure are administered intravenously.

In an example, the compositions described herein may be administered as a single dose.

In some examples, the compositions described herein may be administered over multiple doses. For example, at least 2, at least 3, at least 4, at least 5, at least 6, at least 7, at least 8, at least 9, at least 10 doses, at least 12 doses, at least 14 doses, at least 16 doses. In an example, 8 doses are administered. In another example, 16 doses are administered.

In an example, MLPSCs may be administered intravenously twice per week for four weeks. In another example, MLPSCs are administered once weekly. For example, MLPSCs can be administered once weekly every two weeks. In an example, MLPSCs can be administered once monthly. In an example, 2×10⁶ cells/kg are administered twice per week for four consecutive weeks. In another example, 2×10⁶ cells/kg are administered are administered twice per week for eight consecutive weeks. In this example, the additional dosing is dependent on GvHD symptoms remaining unresolved.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

The present application claims priority from U.S. 63/579,213 filed 28 Aug. 2023, the disclosures of which are incorporated herein by reference.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### EXAMPLES

### Composition

The composition comprises ex-vivo cultured adult human mesenchymal stromal cells (MSCs) derived from bone marrow aspirates. The composition is cryopreserved in Plasma-Lyte® A supplemented with human serum albumin (5%) and dimethyl sulfoxide (10%)). The composition is stored and distributed in vials (25×10⁶ MSCs in 3.8 mL [6.68×10⁶ cells/mL]).

### MLPSC as Third Line Therapy for GvHD

Acute GVHD that has failed to respond to BOTH steroid treatment and a second-line treatment (in adults the only approved second line treatment being ruxolitinib) remains a significant unmet clinical need. Such patients, denoted here as SR+ acute GVHD, account for 45% of all adults who receive ruxolitinib as second-line treatment for steroid-refractory acute GVHD, as shown in the pivotal REACH 1 trial data that supported FDA approval for ruxolitinib in those aged 12 and older (Jagasia et al., (2020) Blood, 135:1739-1749). In addition, as shown in the REACH 2 trial, by 56 days post ruxolitinib initiation as second-line therapy as many as 61% of patients fail to maintain a treatment response and 32% of patients are intolerant to the drug due to treatment ineffectiveness or toxicity (Zeiser et al., (2020) N Engl J Med., 382:1800-1810). Most importantly, survival in these ruxolitinib-resistant patients was only approximately 30% at 3 months post ruxolitinib initiation (Jagasia et al 2020). Recently published results from a retrospective multi-center study of real-world experience with ruxolitinib as second-line treatment for steroid refractory acute GVHD found a median survival of only 21 days for 48 patients who were resistant to ruxolitinib and 50 days for an additional 16 patients who were intolerant to ruxolitinib. Median survival for this 64-patient group of ruxolitinib-resistant and/or intolerant patients was 28 days (interquartile range 7-90), with only 25% overall survival by 3 months (Abedin et al., (2021) British Journal of Haematology, 195:429-43). For 33 patients who received third-line treatment with best available therapy in this group, day 28 overall response rate (ORR) was 36% and median survival was 28 days (Abedin et al., 2021). These data point to and highlight a clear need in this patient population for safe and effective therapies that result in improved responses and survival.

71 patients aged 12 and older with acute GvHD who failed to respond to treatment with both steroids and at least one other GvHD treatment received mesenchymal stem cells under a prospectively-defined protocol as third-line treatment.

The 71 patients come from two patient cohorts. The first cohort (cohort 1) consists of patients aged 12 or older and is made up of 49 patients treated with MSCs. The second cohort (cohort 2) comprises 22 patients (paediatric and adult) also treated with MSCs. 28 Overall Response Rate (ORR) and Overall Survival (OS) through 100 days was assessed in both cohorts. Despite being refractory to two prior lines of therapy, administration of MSCs as third line therapy resulted in a 28-day ORR of 59% and OS through 100 days was 63%.

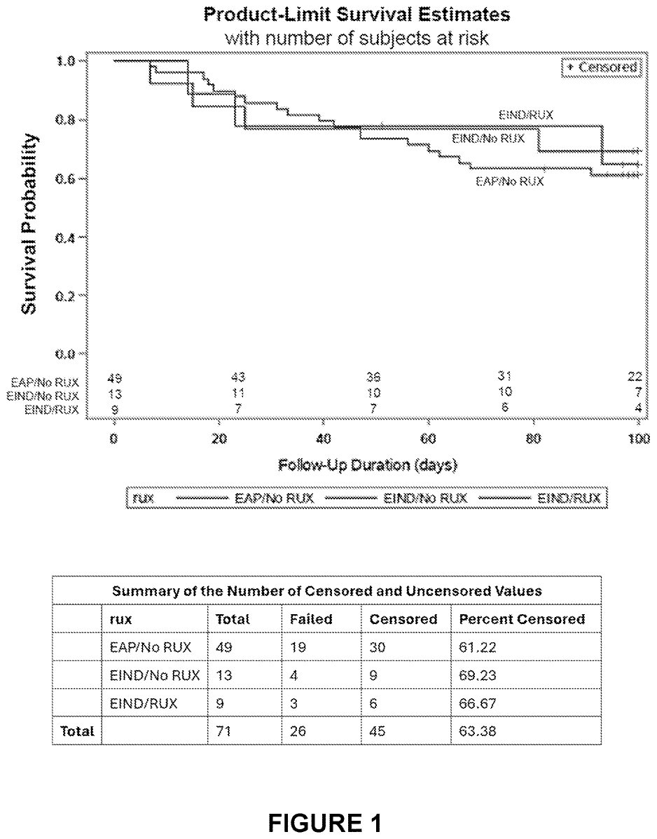

As shown in FIG. 1, survival through 100 days is 61% for cohort 1 (EAP/NoRUX), 69% for the 13 patients from cohort 2 (9/13) who did not receive ruxolitinib as second-line (EIND/NoRUX), and 67% for 9 patients (6/9) from cohort 2 who did receive ruxolitinib as second-line (EIND/RUX). Therefore 63% overall day 100 survival was observed in patients who received MSCs as third-line treatment (45/71) under a prospectively-defined protocol.

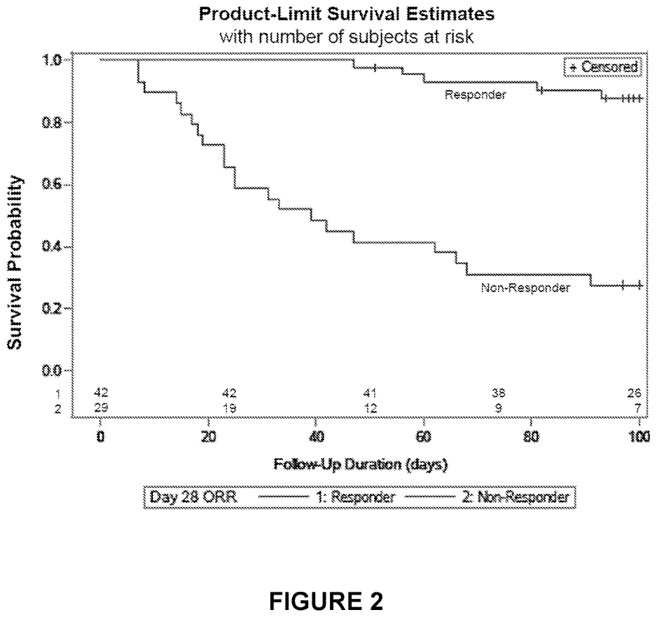

Similarly, the 59% day 28 ORR for the 71 patients reflected rates of 57%, 62% and 67% for the three subgroups, all substantially higher than the 36% reported with best available third-line therapy in ruxolitinib-resistant patients (Abedin et al 2021). Among those MSC treated patients who achieved a Day 28 ORR, 88% (37/42) were alive at day 100 compared with only 28% (8/29) of those who did not achieve a Day 28 ORR (p<0.0001), FIG. 2. A landmark survival analysis for day 100 survival only for those patients who were still alive at day 28 demonstrated that day 100 survival was 88% in those with Day 28 ORR and 47% for those without a response (log-rank p=0.003), confirming that the Day 28 ORR intermediate endpoint predicts survival benefit in this patient population treated with MSCs.

These results suggest that MSCs may be a highly effective third-line treatment for patients with steroid refractory GVHD and could significantly improve response rates and survival in these patients at very high risk of early mortality.

### Comparison to Second Line Treatment

Table 4 shows the primary endpoint of Day 28 Overall Response in children aged 18 and under with SR-aGVHD and treated with MSCs as either second-line after failure of corticosteroids alone (n=54) or as third-line after failure of corticosteroids and either ruxolitinib or other agents (n=35).

TABLE 4
Day 28 Overall Response (OR) in MSC
Treatment of Pediatric SR-aGVHD

| Indication | Second-line aGVHD | Third-line aGVHD |
|---|---|---|
| N Subjects | 55 | 35 |
| Day 28 OR: N (%) | 38 (69.1) | 22 (62.9) |
| 95% CI: (LI %, UL %) | (55.9, 79.8) | (46.3, 76.9) |
| Null Hypothesis % | 45 | 45 |

Note:
95% CI calculated using the modified Wald method.

For the children who received MSCs as third-line therapy, Day 28 Overall Response was 63% (22/35), with 95% CI: 46.3, 76.9. This was comparable to the 69% Day 28 Overall Response (OR) in children who received MSCs as second-line therapy in (95% CI 55.9, 79.8). Of the third-line therapy children who achieved a day 28 response with MSCs, 20/22 (91%) were alive at day 100 (p=0.0004).

The lower boundary of the 95% CI of 46.3% for MSCs as third-line therapy excluded the prespecified null hypothesis of 45% for determining the MSC product's efficacy as second-line therapy in children under age 18 (Table 1). The 63% Day 28 OR also compared favorably with the 36% that has been published for other treatment agents used as third-line in ruxolitinib-refractory patients in the Abedin et al. article (discussed above).

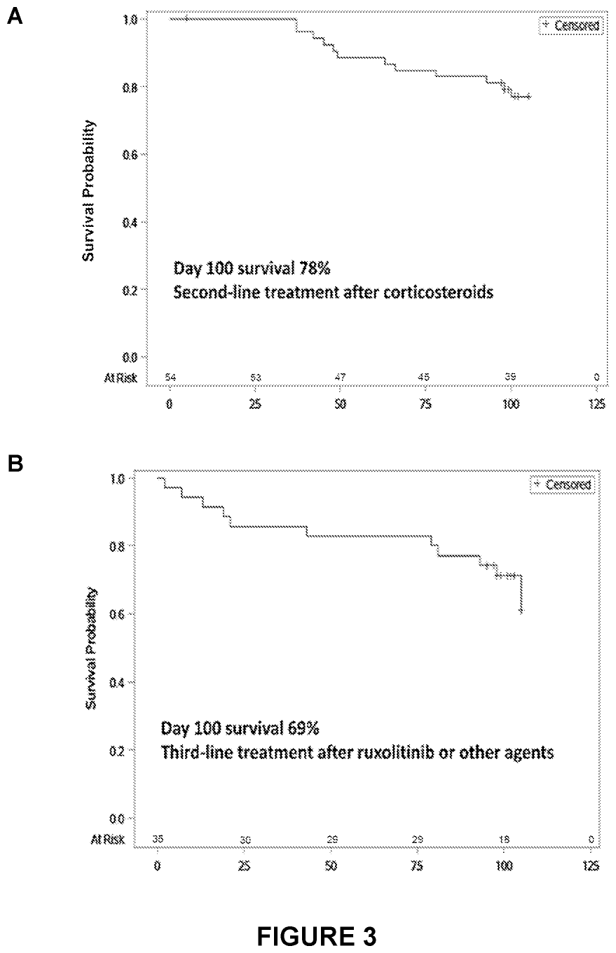

FIG. 3 shows day 100 overall survival in children aged 18 and under with SR-aGVHD and treated with MSCs as either second-line after failure of corticosteroids alone (n=54) or as third-line after failure of corticosteroids and either ruxolitinib or other agents (n=35).

Day 100 overall survival was 78% with MSCs when used as second-line and 69% when used as third-line. In the third-line study, mortality from GVHD-related disease or treatment (deaths due to aGVHD, infections or tumor relapse) at day 100 was 31% (11/35) for those receiving MSCs as third-line agent, compared with 73% for the same mortality analysis in ruxolitinib non-responders in the paper by Abedin et al.

Thus, in two single-arm studies in children aged 18 and under with SR-aGVHD, treatment with MSCs resulted in each trial achieving day 28 Overall Responses that excluded the null hypothesis and in comparably high Day 100 survival rates when MSCs was used as either second-line and as third-line therapy.

### REFERENCES

Hartwell et al., (2017) JCI Insight; 2(3):e89798.
Glucksberg et al., (1974); Transplantation; 18, 295-304.
MacMillan et al., (2015) Biol Blood Marrow Transplant; 21(4): 761-767.
Major-Monfried et al., (2018) Blood; 131(25): 2846-2855.
Rowlings et al., (1997) British Journal of Haematology; 97, 855-864.
Thomas et al., (1975) New England Journal of Medicine; 292, 895-902.

1. A method of treating Graft versus Host Disease (GvHD) in a human subject in need thereof, the method comprising administering to the subject a composition comprising mesenchymal lineage precursor or stem cells (MLPSCs), wherein the MLPSCs are administered to the subject as a third line of therapy.

2. A method for treating Graft versus Host Disease (GvHD) in a human subject, the method comprising:
i) selecting a subject with GvHD that has failed two lines of therapy;
ii) administering to the subject a composition comprising mesenchymal lineage precursor or stem cells (MLPSCs), wherein the MLPSCs are administered to the subject as a third line of therapy.

3. The method of claim 1 or claim 2, wherein the subject is refractory to steroids and a second line of therapy.

4. The method of claim 3, wherein the second line of therapy is ruxolitinib.

5. The method of claim 3, wherein the second line of therapy is a biologic.

6. The method of claim 5, wherein the biologic is alemtuzumab, basiliximab or tocilizumab.

7. The method according to any one of claims 1 to 6, wherein the MLPSCs in the composition inhibit IL2-Rα expression by ≥60%, wherein inhibition of IL2-Rα expression is determined by: obtaining a population of cells comprising culture expanded, cryopreserved and thawed MLPSCs; co-culturing the MLPSCs in a culture medium with a population of cells comprising T cells; and determining the level of inhibition of IL2-Rα expression.

8. The method according to any one of claims 1 to 7, wherein treatment reduces the risk of mortality in the subject.

9. The method of claim 8, wherein mortality is 6-month non-relapse mortality (NRM).

10. The method according to any one of claims 1 to 9, wherein the subject is a paediatric subject.

11. The method according to any one of claims 1 to 9, wherein the subject is ≥12 years of age.

12. The method according to any one of claims 1 to 11, wherein the subject has acute Graft versus Host Disease (aGvHD).

13. The method according to any one of claims 1 to 12, wherein the subject is classified as one or more of the following:
Grade B, C, or D according to the IBMTR severity scale;
Grade II GvHD according to the Glucksberg severity scale;
Grade III/IV GvHD according to the Glucksberg severity scale;
Minnesota high risk GvHD.

14. The method according to any one of claims 1 to 13, wherein the subject has chronic GvHD.

15. The method according to any one of claims 1 to 14, wherein the subject has multi-organ involvement.

16. The method according to any one of claims 1 to 15, wherein the subject has inflammatory bowel disease (IBD).

17. The method of claim 16, wherein the IBD is Crohn's disease or ulcerative colitis.

18. The method of claim 16, wherein the IBD is Crohn's disease.

19. The method of claim 18, wherein the Crohn's disease presents in the rectum and/or colon of the subject.

20. The method according to any one of claims 16 to 19, wherein the subjects IBD is refractory to one or more of adalimumab, certolizumab pegol, vedolizumab or Ustekinumab.

21. The method according to any one of claims 1 to 20, wherein treatment increases the probability of the subject surviving for at least 28 days after initiation of treatment.

22. The method according to any one of claims 1 to 20, wherein treatment increases the probability of the subject surviving for at least 100 days after initiation of treatment, preferably at least 180 days.

23. The method according to claim 21 or 22, wherein the probability of the subject surviving is greater than 40%, greater than 50%, or, preferably, greater than 60% after initiation of treatment.

24. The method according to any one of claims 21 to 23, wherein the subject's probability of survival is increased relative to a subject who does not receive MLPSCs.

25. The method according to any one of claims 1 to 24, wherein the MLPSCs are culture expanded from a population of MLPSCs that are STRO-1+.

26. The method of claim 25, wherein the population of MLPSCs that are STRO-1+ comprises about 0.1% to 75% STRO-1+ cells.

27. The method according to any one of claims 1 to 26, wherein the MLPSCs are mesenchymal stem cells (MSCs).

28. The method according to any one of claims 1 to 27 which comprises administering 2×10⁶ cells/kg.

29. The method according to any one of claims 1 to 27 which comprises administering between 1×10⁷ and 2×10⁸ cells.

30. The method according to any one of claims 1 to 29, wherein the composition further comprises Plasma-Lyte A, dimethyl sulfoxide (DMSO), human serum albumin (HSA).

31. The method according to any one of claims 1 to 30, wherein the composition comprises greater than 6.68×10⁶ viable cells/mL.

32. The method according to any one of claims 1 to 31, wherein the composition is administered intravenously.

33. The method according to any one of claims 1 to 32, wherein the subject receives at least two doses.

34. The method according to claim 33, wherein the first two doses are administered twice weekly for four weeks.
